(19) United States
(12) Patent Application Publication
(10) Pub. No.: US 2023/0183748 A1
(43) Pub. Date: Jun. 15, 2023

(54) Use of FOXP3 Enhancers to Modulate Regulatory T Cells

(71) Applicant: St. Jude Children's Research Hospital, Inc., Memphis, TN (US)

(72) Inventors: Yongqiang FENG, Memphis, TN (US); Xinying ZONG, Memphis, TN (US)

(21) Appl. No.: 17/550,051

(22) Filed: Dec. 14, 2021

(51) Int. Cl. C12N 15/90; A61K 35/17

(52) U.S. Cl. CPC C12N 15/907; A61K 35/17; C12N 2310/20

(57) ABSTRACT

Disclosed are methods for modifying regulatory T cell lineage stability or induction by simultaneously modulating the activity of Foxp3 enhancers, CNS0 and CNS2 or CNS0 and CNS3. Methods for treating cancer, an autoimmune disease or condition, or a regulatory T cell-related disease or condition are also provided.

### INTRODUCTION

This invention was made with government support under grant nos. AI153138 and AI146614 awarded by the National Institutes of Health. The government has certain rights in this invention.

### BACKGROUND

Regulatory T (Treg) cells with a diverse T cell receptor (TCR) repertoire are induced by tissue environmental cues (e.g., TCR agonists, IL-2, and TGF-β) in the thymus and periphery to suppress effector T cells (TE) that not only cause autoimmune diseases but also elicit antitumor immune response. Upon commitment, Treg cells rely on several pathways, including IL-2 and TCR signaling, to maintain their lineage identity, fitness, and immune suppressive function during their long lifespan. In addition, a growing list of cell-intrinsic and -extrinsic factors have been discovered to modulate Treg cell development and function, thus fine-tuning immune tolerance.

Treg cell induction and lineage maintenance are governed by cues that fluctuate. Therefore, stochasticity affects Treg cell clones for specific antigens or epitopes. Immune perturbations and genetic variations may further shape the Treg cell TCR repertoire, impairing immune tolerance. These adversities raise a question about the mechanisms by which a diverse TCR repertoire and stable lineage identity are programmed at the genetic and epigenetic levels to confer on Treg cells an adequate suppressive capacity that opposes the uncertainties during Treg cell development and lineage maintenance.

Investigation of the mechanisms governing the expression of Treg cell lineage has indicated an involvement by winged-helix transcription factor forkhead box P3 (Foxp3) (Josefowicz et al. (2012) Annu. Rev. Immunol. 30: 531-564; Sakaguchi et al. (2020) Annu. Rev. Immunol. 38:541-566). In particular, Foxp3 enhancers have been characterized as individual modules that integrate distinct environmental cues and epigenetic programs to dictate Treg cell development or lineage stability. Ablation of conserved noncoding sequence (CNS) 1 compromises immune tolerance at the mucosal surfaces because of impaired peripheral Treg cell development (Campbell et al. (2018) Immunity 48:1245-1257.e9; Josefowicz et al. (2012) Nature 482:395-399; Samstein et al. (2012) Cell 150:29-38); CNS2 deficiency destabilizes heritable Foxp3 expression and Treg cell fate in adverse environments, causing chronic tissue inflammation in aged mice or after immune challenge (Feng et al. (2014) Cell 158:749-763; Li et al. (2014) Cell 158:734-748); and loss of CNS0 or CNS3 reduces Treg cell development in both the thymus and periphery, yet without causing noticeable immune dysregulation (Dikiy et al. (2021) Immunity 54:931-946.e11; Feng et al. (2015) Nature 528:132-136). However, in the absence of autoimmune regulator Aire, a gene required for both thymic Treg cell induction and negative selection by facilitating the expression of tissue-restricted antigens in medullary thymic epithelial cells (Klein et al. (2014) Nat. Rev. Immunol. 14:377-391), CNS0 or CNS3 deficiency abruptly disrupts Treg cell-mediated immune tolerance, leading to early-onset, multiorgan autoimmunity, in contrast to Aire single-deficient mice. Moreover, deletion of both CNS0 and CNS3 causes lethal autoimmunity (Kawakami et al. (2021) Immunity 54(5):P947-961).

Manipulation of Treg cell function for therapeutic purposes has been a major goal in immunotherapy. Most methods use growth factors and cytokines to expand Treg cells or improve their function. However, because Treg cells are flexible in their lineage identity, these methods have very limited potential in treating autoimmune diseases because once Treg become effector T cells they exacerbate disease pathology. In addition, Treg cells are extremely diverse in their antigen specificity. Accordingly, alternative approaches for manipulating Treg cell function are needed.

### SUMMARY OF THE INVENTION

This invention provides a method for modifying Treg cell lineage stability by simultaneously modulating the activity of conserved noncoding sequence (CNS) 0 and CNS2 of the forkhead box P3 (Foxp3) locus in a Treg cell. In one aspect, Treg cell lineage is destabilized, e.g., by reducing CNS0 and CNS2 activity via one or more protein factors that bind CNS0 and CNS2, CNS0 and CNS2 silencing (e.g., by recruiting transcriptional or epigenetic inhibitory protein factors to CNS0 and CNS2 via CRISPR-based or TALEN-based approaches), or inhibiting chromatin looping of CNS0 and CNS2 with the Foxp3 promoter. In another aspect, Treg cell lineage stability is enhanced, e.g., by increasing CNS0 and CNS2 activity via one or more protein factors that bind CNS0 and CNS2, recruiting transcriptional or epigenetic activating protein factors to CNS0 and CNS2 via CRISPR-based or TALEN-based approaches, insertion of at least one exogenous CNS0 and CNS2 nucleic acid molecule into the Foxp3 locus, or by increasing chromatin looping of CNS0 and CNS2 with the Foxp3 promoter. In accordance with targeting CNS0 and CNS2 to modify Treg cell lineage stability, this invention also provides a method for treating cancer by (a) reducing the activity of CNS0 and CNS2 in a Treg cell; and (b) administering the Treg cell of (a) to a subject with cancer. Moreover, this invention provides a method for treating an autoimmune disease or condition by (a) increasing the activity of CNS0 and CNS2 in a Treg cell; and (b) administering the Treg cell of (a) to a subject with an autoimmune disease or condition.

This invention also provides a method for modifying Treg cell induction by simultaneously modulating the activity of CNS0 and CNS3 in a Treg cell precursor, e.g., a naïve CD4 T cell, hematopoietic stem cell, or lymphoid progenitor cell. In one aspect, CNS0 and CNS3 activity is increased via one or more protein factors that bind CNS0 and CNS3, insertion of at least one exogenous CNS0 and CNS3 nucleic acid molecule into the Foxp3 locus, recruiting transcriptional or epigenetic activating protein factors to CNS0 and CNS3 via CRISPR-based or TALEN-based approaches, or by increasing chromatin looping of CNS0 and CNS3 with the Foxp3 promoter. In accordance with targeting CNS0 and CNS3 to modify Treg cell induction, this invention also provides a method for treating a Treg cell-related disease or condition caused by impaired regulatory T cell diversity or coverage by (a) increasing the activity of CNS0 and CNS3 in a regulatory T cell precursor; and (b) administering the regulatory T cell precursor of (a) to a subject in need of treatment.

### BRIEF DESCRIPTION OF THE DRAWINGS

FIG. 1 shows validation of the effects of sgRNAs targeting CNS1 and CNS3 on induced Treg (iTreg) cell development. The percentages of GFP-Foxp3⁺ among live transduced cells were normalized to those of sgNC (nontargeting control)-transduced cells of the same genotypes (left y-axis). The ratios of normalized GFP-Foxp3⁺ percentages of ΔCNS0 cells to those of WT cells were calculated (right y-axis). Technical replicates are shown. sgCNS1 and sgCNS3 represent three experiments, and sgCNS2-1 represents one experiment. The differences between WT and ΔCNS0 were compared between sgCNS2-1 and other sgRNAs by two-way ANOVA. *, P<0.05; **, P<0.01.

FIG. 2 shows validation of the effects of sgRNAs targeting CNS0 on Foxp3 expression in ASC-treated iTreg cells. Results were analyzed as described in FIG. 1. Data show technical replicates and represent three experiments. ***, P<0.001; ****, P<0.0001.

FIG. 3 shows Treg cell induction in vitro. CD4 Tn cells sorted from mixed bone marrow chimeric mice were used for iTreg cell induction. Frequencies of GFP-Foxp3⁺ cells are shown. Data are mean±SEM of triplicates and represent two experiments. *, P<0.05; ***, P<0.001 by unpaired t test.

FIG. 4 depicts the experimental procedures for assessing the stability of Foxp3 expression. CD4 Tn cells from mixed bone marrow chimeras were cultured in Treg cell-inducing conditions with or without ASC. iTreg cells were sorted and cultured in fresh media for 4 days before analysis.

FIG. 5 illustrates how CNS3 and CNS0 indirectly or directly integrate TCR and IL-2/Stat5 signaling, respectively, to promote Foxp3 induction. Pro, promoter.

FIG. 6 is a model of CNS0 and CNS3 activity in determining Treg cell induction efficiency and Treg cell suppressive capacity.

FIG. 7 shows that CNS0 and CNS2 coordinate to confer stable T reg cell lineage identity. Shown are the ratios of Treg cells in the periphery versus those in the thymus of the same mice. n=6 per genotype. Data represent two experiments.

FIG. 8 illustrates how CNS2 is activated by Tet-induced DNA demethylation, and CNS0 is constitutively hypomethylated.

FIG. 9 depicts the coordination between CNS0 and CNS2 to maximize T reg cell lineage stability during activation and proliferation.

FIG. 10 shows a model of CNS0 and CNS2 involvement in T reg cell suppressive capacity against genetic variations and immune perturbations.

### DETAILED DESCRIPTION OF THE INVENTION

To understand the mechanisms sustaining immune tolerance in CNS0-, CNS2-, and CNS3-deficient mice, the regulatory space and modes of Foxp3 enhancers were explored. Using CRISPR screening and genetic perturbations, substantial synergies were observed among these enhancers during Treg cell induction and lineage maintenance. Based upon these analyses, it has now been shown that there is a broad coordination of cell-intrinsic and -extrinsic factors acting through Foxp3 enhancers to control Treg cell suppressive capacity by modulating Treg cell induction or lineage stability, thus determining the robustness of Treg cell-dependent immune tolerance. Specifically, it has now been found that enhancers CNS0 and CNS2 regulate Treg cell lineage stability, whereas enhancers CNS0 and CNS3 regulate Treg cell induction probabilities.

Accordingly, this invention provides methods for manipulating Treg cell function by simultaneously targeting CNS0 and CNS2 to modify Treg cell lineage stability, or simultaneously targeting CNS0 and CNS3 to modify Treg cell induction probabilities. Such methods find use in effectively enhancing or dampening Treg cell function, thereby overcoming the natural potency of Treg cells in treating many diseases including but not limited to autoimmune diseases, metabolic dysregulation, tissue regeneration, age-related neurodegeneration, and cancer, where Treg cells play important and distinct roles.

Foxp3 (forkhead box P3), also known as scurfin, is a protein involved in immune system development and function (Brunkow et al. (2001) Nat. Genet. 27(1):68-73). A member of the Fox protein family, FoxP3 functions as a master regulator of the development and function of regulatory T cells (see, e.g., Hon et al. (2003) Science 299 (5609):1057-1061; Fontenot et al. (2003) Nat. Immunol. 4:330-336; Fontenot et al. (2005) Immunity 22:329-341). The locus encoding Foxp3 as well as the Foxp3 protein sequences are well known in the literature and available from sources such as the National Center for Biotechnology Information (NCBI) database. See, for example, NCBI entries: Accession Nos. NM_001114377, NM_014009, NG_007392, XM_006724533, XM_017029567, NP_001107849, NP_054728, XP_016885056, and XP_006724596.

As used herein, the “Foxp3 locus” refers to the Foxp3 coding sequence as well as nucleotide sequences upstream of, downstream of or intervening the coding sequence, which control or regulate the transcription or translation of the Foxp3 coding sequence. In particular aspects, the Foxp3 locus includes, the Foxp3 promoter, Foxp3 coding sequence, introns, and enhancers such as conserved noncoding sequences (CNSs). CNS0 is located upstream of the Foxp3 promoter. CNS1, CNS2, and CNS3 are in the introns of the Foxp3 locus. These enhancer elements are cis-acting transcriptional regulatory elements, a.k.a. cis-elements, which confer an aspect of the overall modulation of gene expression.

CNS0, which is located approximately 8000 bp upstream of the Foxp3 transcription start site in an intron of the neighboring gene, includes a Satb1 (special AT-rich sequence binding protein 1) binding site (Kitagawa et al. (2017) Nat. Immunol. 18:173-183). Satb1 binds CNS0 and triggers Treg cell differentiation due to Foxp3 induction. Disruption of Satb1-mediated enhancer activation not only leads to impaired Foxp3 induction but also diminishes DNA demethylation in Treg-specific demethylation regions (Kitagawa et al. (2017) Nat. Immunol. 18:173-183). CNS0 also functions as an IL-2/STAT5 (signal transducer and activator of transcription 5) response element (Akamatsu et al. (2019) Sci. Immunol. 4:eaaw2707; Dikiy et al. (2021) Immunity 54:931-946.e11) enabling Foxp3 induction in differentiating and mature Treg cells (Dikiy et al. (2021) Immunity 54:931-946.e11). The nucleotide sequence of CNS0 is:

(SEQ ID NO: 1)
GAAAAGGGATCCCCTGAGGTCCACCACCATTTCCC
CAGAGGGCTGGATCACGGGGGGTAGCTATTCTTCA
ACAGCACTTCAAATCAGCAGCAGCACACAGGCCTT
AAAACAATAATAAGTTGAAATGTATTTGCTAGGAA
AGTCACCGACCTACAAAGAAAACCTTATCGCTGAT
CTAGCAGCGCACACCAGCCTCCCCTTTGCAAGAGC
TGAGATCAAAAGATAAAGAAGCTATCAAAAAGCCA
TCTGCCCACTTAAAATAACATCTCAAGTCACGTTG
GGAACCACAAACATGGGGCCAGCTACCAAAACAAT
TGTCTAAATGAACTACTTCAATTTCTCCTTAAAAC
CACCCATGTATTTTAAAAGAAAAACACCCTCTCCA
CCCACCTTGGCACGGCAAGGTTTTGATTTGTCTGT
TCCCTTCCTTTCACATTCTTGAAAATGACCAAACT
TCAGTACTCAACTGTCTTATCTTCCAGAAAGGGCT
CCCACAACTG.

CNS2, which is located approximately 4000 bp downstream of the Foxp3 transcription start site between exon −2b and −1, is described by Kim & Leonard ((2007) J. Exp. Med. 204:1543-1551). CNS2 has been shown to maintain heritable Foxp3 expression. Like CNS0, CNS2 contains Treg cell-specific STAT5 binding sites and also contains CREB (cAMP response element binding protein) binding sites (Kim & Leonard (2007) J. Exp. Med. 204:1543-1551). Further, STAT3 binding to the CNS2 region has been shown to inhibit Fox3p gene transcription (Zorn et al. (2006) Blood 108(5):1571-9). Moreover, Foxp3 binds to CNS2 in a CBF-β-Runx1 (core binding factor-β-runt-related transcription factor 1)-dependent and CpG DNA demethylation-dependent manner. Additional transcription factors that have been shown to bind to CNS2 include Mbd2 (methyl-CpG-binding domain 2), Tet2 (Tet methylcytosine dioxygenase 2), Eno-1 (Enolase 1), and Foxp3-E2 (Forkhead box-p3-Exon2) (Colamatteo et al. (2019) Front. Immunol. 10:3136). The nucleotide sequence of CNS2 is:

(SEQ ID NO: 2)
GGGTTTTGCATGGTAGCCAGATGGACGTCACCTAC
CACATCCGCTAGCACCCACATCACCCTACCTGGGC
CTATCCGGCTACAGGATAGACTAGCCACTTCTCGG
AACGAAACCTGTGGGGTAGATTATCTGCCCCCTTC
TCTTCCTCCTTGTTGCCGATGAAGCCCAATGCATC
CGGCCGCCATGACGTCAATGGCAGAAAAATCTGGC
CAAGTTCAGGTTGTGACAACAGGGCCCAGATGTAG
ACCCCGATAGGAAAACATATTCTATGTCCCAGAAA
CAACCTCCATACAGCTTCTAAGAAACAGTCAAACA
GGAACGCCCCAACAGACAGTGCAGGAAGCTGGCTG
GCCAGCCCAGCCCTCCAGGTCCCTAGTACCACTAG
ACAGACCATATCCAATTCAGGTCCTCTTTCTGAGA
ATGTACTGATGCATCACACAGTCACACCAGTTCCA
CAAGTATTTAAGGAGGAGATTTCTTATAAGTTCTG
ACCAAACATAAAGAGCACTTCAAAAGTGA.

CNS3, located approximately 7000 bp downstream of the Foxp3 transcription start site, has been shown to be involved in thymic induction of Foxp3 expression. c-Rel binds to the CNS3, which exists in an open chromatin configuration, to facilitate Foxp3 gene transcription and nTreg generation (Zheng et al. (2010) Nature 463(7282):808-812). Additional transcription factors that have been shown to bind to CNS3 include Satb1, Forkhead transcription factor of the O class (Foxo)1 and Foxo3 (Colamatteo et al. (2019) Front. Immunol. 10:3136). The nucleotide sequence of CNS3 is:

(SEQ ID NO: 3)
GTGAGGCCCGGGGCCCAGAATGGGGTAAGCAGGGT
GGGGTACTTGGGCCTATAGGTGTCGACCTTTACTG
TGGCATGTGGCGGGGGGGGGGGGGGGGGCTGGGGC
ACAGGAAGTGGTTTATGGGTCCCAGGCAAGTCTGA
CTTATGCAGATATTGCAGGGCCAAGAAAATCCCCA
CTCTCCAGGCTTCAGAGATTCAAGGCTTTCCCCAC
CCCTCCCAATCCTC.

As demonstrated by the results presented herein, the simultaneous modulation of the activity of CNS0 and CNS2 or CNS0 and CNS3 results in the synergistic modification of Treg lineage stability and Treg induction, respectively. Accordingly, this invention provides methods for the coordinate modulation of CNS0 and CNS2 activity or coordinate modulation of CNS0 and CNS3 activity. Coordinate modulation is achieved by simultaneous or substantially simultaneous modulation of CNS0 and CNS2 activity or CNS0 and CNS3 activity. For the purposes of this invention, “simultaneous” or “substantially simultaneous” refers to immediately or nearly immediately in time. “Modulation” in the context of this invention encompasses a reduction or increase in the activity of CNS0, CNS2 and CNS3. As used herein, the term “activity,” with regard to CNS0, CNS2 and CNS3, refers to the ability of said enhancer elements to affect the expression pattern of Foxp3, by affecting the transcription of Foxp3. Gene regulatory activity may be positive and/or negative and the effect may be characterized by Treg cell function, Treg cell frequencies, precursor-to-Treg cell transition, TCR diversity intensities, or Treg cell lineage stability, as well as by quantitative or qualitative efficiency, expression level, or maintenance of Foxp3 expression.

Simultaneous increase or reduction in enhancer element activity can be achieved via manipulating one or more protein factors that bind the enhancer element, insertion or silencing of the enhancer element, recruiting transcriptional or epigenetic activating or inhibitory protein factors to these enhancer elements via CRISPR-based or TALEN-based approaches, enhancing or inhibiting chromatin looping of the enhancer element with the Foxp3 promoter, or a combination thereof.

As described herein, there are a number of protein factors known to bind to CNS0, CNS2 and CNS3. Specifically, Satb1, IL-2 and STAT5 bind to CNS0; STAT5, CREB, STAT3, CBF-β-Runx1, Mbd2, Tet2, Eno-1, and Foxp3 bind to CNS2; and c-Rel, Satb1, Foxo1, and Foxo3 bind the CNS3. The expression or activity of one or more of these protein factors can be increased or decreased thereby respectively increasing or decreasing enhancer element activity. In some aspects, protein factor expression is increased by inserting one or more exogenous nucleic acid molecules encoding the protein factor to increase copy number. Methods for the ex vivo or in vivo introduction of exogenous nucleic acid molecules to produce recombinant cells is well known in the art and can include standard transient expression vectors, retroviruses, lentivirus-based vectors, or endonucleases, such as CRISPR-associated (CRISPR/Cas9, Cpfl, and the like) nucleases to insert the exogenous nucleic acid sequence into a genomic locus of the cells. By way of illustration, a recombinant cell can be prepared by providing a cell (e.g., a Treg or Treg precursor), introducing a CAS9 protein or nucleic acid encoding a CAS9 protein into the cell, introducing a second nucleic acid encoding at least one CRISPR guide sequence or a set of nucleic acids encoding at least one CRISPR guide sequence, wherein the at least one CRISPR guide sequence is configured to hybridize to at least one targeted locus in the cell and introducing a third nucleic acid into the cell, wherein the third nucleic acid is a gene delivery cassette harboring the exogenous nucleic acid of interest.

In other aspects, protein factor expression or activity is decreased with an inhibitor such as an antisense oligonucleotide, a siRNA/shRNA olignonucleotide, a small molecule that interferes with protein factor function, a viral vector producing a nucleic acid molecule that inhibits the protein factor, an aptamer or gene editing. See, e.g., U.S. Pat. Nos. 9,884,863 B2, 9,873,674 B2, US 2008/0280298 A1, US 2014/0206624 A1, US2021/0205292 A1, US 2020/0362308 A1, WO 2014/068397 A1, WO 2015/194710 A1, WO 2021/081448 A1, and WO 2020/169707 A1.

In a further aspect, regulatory T cell lineage stability or induction is modified by insertion or silencing of one or more of CNS0, CNS2 and/or CNS3. Insertion of one or more enhancer elements can be carried out by inserting one or more exogenous CNS0, CNS2 and/or CNS3 nucleic acid molecules into the Foxp3 locus thereby affecting an increase in Foxp3 expression. Methods for the ex vivo or in vivo introduction of one or more exogenous CNS0, CNS2 and/or CNS3 nucleic acid molecules into a cell can include the use of standard CRISPR-associated nucleases to insert the exogenous nucleic acid sequence into the Foxp3 locus of the cells. Conversely, silencing (e.g., by disruption, excision or replacement) of endogenous CNS0, CNS2 and/or CNS3 nucleic acid molecules (e.g., by gene editing) can be used to decrease Foxp3 expression.

In another aspect, regulatory T cell lineage stability or induction is modified by activating or inhibiting one or more of CNS0, CNS2 and/or CNS3, by recruiting transcriptional or epigenetic activating or inhibitory proteins to these enhancer elements. This can be achieved by genome editing methods, e.g., CRISPR-mediated or TALEN-mediated targeting approaches. For example, the CRISPR-mediated targeting of transcriptional or epigenetic activating or inhibitory proteins to these enhancer elements can be achieved by guide RNAs (gRNAs) targeting the respective enhancer elements in combination with dCas9 (a mutant form of Cas9 whose endonuclease activity is removed through point mutations in its endonuclease domains) fused to transcriptional or epigenetic activating or inhibitory proteins or their functional domains, e.g., dCas9-Krab, dCas9-VP64, dCas9-p300, or dCas9-catalytic core of p300. See, e.g., Hilton et al. (2015) Nat. Biotechnol. 33(5):510-7.

In yet another aspect, chromatin looping of the enhancer element with the Foxp3 promoter can be modified. The human genome is partitioned into domains that are associated with particular patterns of histone marks that segregate into sub-compartments, distinguished by unique long-range contact patterns. A loop domain is a domain whose endpoints are anchored to form a chromatin loop. Loops are anchored at DNA sites bound by higher-order loop anchor complexes containing loop anchor proteins such as CTCF and cohesin, and other factors. The pairs of these motifs including CTCF binding sites that anchor a loop are nearly all found in the convergent orientation. Accordingly, in one embodiment, modification or deletion (e.g., by gene editing) of the chromatin loop anchor or chromatin loop structure results in preventing enhancer associated factors or the mRNA splicing machinery associated with an actively transcribed gene from interacting with a transcription complex, so as to alter Foxp3 transcription or mRNA splicing. In another embodiment, modification or deletion of the chromatin loop anchor or chromatin loop structure results in allowing enhancer associated factors or a mRNA splicing machinery associated with an actively transcribed gene to interact with a transcription complex, so as to alter Foxp3 transcription or mRNA splicing. In a further embodiment, introduction of a new chromatin loop anchor or chromatin loop structure results in allowing enhancer associated protein factors or mRNA splicing machinery to associate with a transcription complex of an actively transcribed gene, so as to alter Foxp3 transcription or mRNA splicing. See, e.g., US 2018/0245079 A1. Alternatively, inhibition of histone acetylation can be used to disrupt the activity of CNS0, CNS2, CNS3 or a combination thereof. Other inhibitors targeting DNA looping mechanisms may also directly affect enhancer activities.

In some aspects, the activity of CNS0 and CNS2 or CNS0 and CNS3 is modified in a Treg cell. “Regulatory T lymphocytes,” “Treg cells,” or “Tregs” are a specialized subpopulation of T cells that act in a “regulatory” way to suppress activation of the immune system and thereby maintain immune system homeostasis and tolerance to self-antigens, food and commensal bacteria, as well as cancer. Treg cells have sometimes been referred to suppressor T-cells. Treg cells are characterized by the expression of forkhead family transcription factor Foxp3. They may express CD4 or CD8 surface proteins. They usually also express CD25 and CTLA-4. As used herein, Treg cells include natural Treg cells and induced or adaptive Treg cells and Treg cells that have been created using recombinant DNA technology. Naturally-occurring Treg cells (CD4⁺CD25⁺Foxp3⁺) arise like all other T cells in the thymus. In contrast, adaptive Treg cells (also known as Tr1 cells or Th3 cells) may originate during a normal immune response but do not express Foxp3, thus not considered as bona fide Treg cells. Antigen-specific activation of human effector T cells leads to inducible and transient expression of Foxp3 in a subgroup of the activated effector cells. These cells are still effector T cells and not Treg cells. One way to induce Treg cells is by prolonged exposure of naïve T cells (Tn cells) to IL-2 and TGF-β during their activation by antigens. T cells may also be converted to Treg cells by transfection or transduction of the Foxp3 gene into a mixed population of T cells. A T cell that is forced to express Foxp3 partially adopts the Treg phenotype and such recombinant Treg cells are also defined herein as “Treg cells”.

Reducing or increasing the activity of CNS0 and CNS2 in a Treg cell is of particular use in modifying Treg cell lineage stability. “Treg cell lineage” refers to Treg cell identity and the ability of Treg cells to lose Foxp3 expression and thereafter acquire effector T cell activities. “Effector T cells” refers to a subset of T cells which are associated with immune response. They are characterized by the secretion of one or more effector cytokines such as, but not limited to, IFN-γ, TNF-α, IL-2, IL4, IL-13, IL-17, and granzyme B. In certain aspects, Treg cell lineage stability is enhanced, i.e., the frequency or number of Treg cells is maintained or increased, by increasing the activity of CNS0 and CNS2. In other aspects, Treg cell lineage is destabilized, i.e., the frequency or number of Treg cells is decreased and optionally effector T cell or effector-like exTreg cell frequency or number increases, by decreasing the activity of CNS0 and CNS2. In accordance with this aspect, a Treg cell is modified ex vivo or in vivo as described above to reduce or increase CNS0 and CNS2 activity.

Methods for the isolation of human Foxp3⁺ Treg cells are known. For example, CD4⁺CD25⁺ T cells with regulatory function can be obtained from standard leukapheresis products by using a 2-step magnetic cell-separation protocol (Hoffmann et al. (2006) Biol. Blood Marrow Transplant 12:267-74). Also, commercial kits, e.g., CD4⁺CD25⁺ Regulatory T Cell Isolation Kit from Miltenyi Biotec or DYNAL® CD4⁺CD25⁺ Treg Kit from Invitrogen are also available.

In other aspects, the activity of CNS0 and CNS2 or CNS0 and CNS3 is modified in a Treg cell precursor. A majority of naturally occurring Treg (nTreg) cells present in the immune system are thymus-derived (thymus-derived Treg or tTreg cells). A proportion of FoxP3⁺ Treg cells differentiate in the periphery from CD4 naïve conventional T (Tn) cells under certain conditions (peripherally derived Treg or pTreg cells). In addition, FoxP3⁺ Treg cells phenotypically similar to tTreg or pTreg cells can be generated in vitro (induced Treg or iTreg cells) from CD4 Tn cells by antigen stimulation in the presence of TGF-β and IL-2. These Treg populations (tTreg, pTreg, and iTreg cells) commonly express FoxP3, CD25, CTLA-4 and other Treg function-associated molecules. Moreover, Treg cells can be produced from pluripotent stem cells, mesodermal stem cells, mesenchymal stem cells, hematopoietic stem cells, or lymphoid progenitor cells (see, e.g., WO 2021/092581 A1).

Reducing or increasing the activity of CNS0 and CNS3 in a Treg cell precursor is of particular use in modifying Treg cell induction. In one aspect, a reduction in CNS0 and CNS3 activity decreases or blocks Treg cell induction as evidenced by, e.g., a decrease in IL-2-induced precursor-to-Treg cell transition. In accordance with this aspect, a Treg cell precursor is modified ex vivo or in vivo as described above to reduce or increase CNS0 and CNS3 activity.

Treg cell precursors of use in this invention can be isolated by any suitable method known in the art. For example, to isolate hematopoietic stem cells (HSCs) from the blood or bone marrow, the cells in the blood or bone marrow may be panned by antibodies that bind unwanted cells, such as antibodies to CD4 and CD8 (T cells), CD45 (B cells), GR-1 (granulocytes), and lad (differentiated antigen-presenting cells) (see, e.g., Inaba et al. (1992) J. Exp. Med. 176:1693-1702). HSCs can then be positively selected by antibodies to CD34.

Treg cells and Treg cell precursors used in accordance with this invention are mammalian cells, such as human cells, cells from a farm animal (e.g., a cow, a pig, or a horse), and cells from a companion animal (e.g., a cat or a dog). The cells may be isolated from a biological sample from a healthy subject or a subject having or suspected of having a disease (e.g., cancer, or an inflammatory and immune dysregulatory condition). The biological sample may be any sample, such as a blood sample, a sample of peripheral blood mononuclear cells (PBMCs), or inflamed tissue. The desired cells can be isolated from a bodily fluid (e.g., blood) or tissue and cultured prior to manipulated in accordance with the methods described herein. Culture media may be optionally supplemented with interleukin 2 (IL-2) and IL-7 to expand the numbers of cells.

The invention is based, at least in part, on the discovery that the CNS0 and CNS2 coordinate to synergistically regulate Treg cell lineage stability, especially during activation and expansion, conferring persistent immune tolerance. In particular, the disclosure is based upon the recognition that Treg suppressive capacity can be reduced by simultaneously silencing enhancers CNS0 and CNS2 or by inhibiting their chromatin looping to the promoter, thus destabilizing Treg lineage identity and enhancing the anti-tumor activity of effector T cells whose function is otherwise tightly constrained by Treg cells. Conversely, Treg cells can be modified to activate enhancers CNS0 and CNS2 thereby enhancing lineage stability and restricting conversion to effector T cells for the treatment of autoimmune diseases. Thus, modulating CNS0 and CNS2 activity in Treg cells is useful for the treatment of certain diseases (e.g., cancers or autoimmune diseases) where therapeutic benefit could be derived from either enhancing or suppressing a subject's immune response.

Treg cells are known to play a role in suppressing effective Th1 responses in tumors. Without wishing to be bound by any particular theory, decreasing the activity level CNS0 and CNS2 induces differentiation of CD4+ Treg into effector T cells, thereby removing the suppressive phenotypes of these T cells. Therefore, methods and compositions described by the disclosure may be useful in the treatment of diseases such as, but not limited to, cancer and infections. In some aspects, the disclosure provides a method for treating cancer in a subject by reducing the activity of CNS0 and CNS2 in a regulatory T cell (e.g., using one or more protein factors that bind CNS0 and CNS2, CNS0 and CNS2 silencing, or inhibiting chromatin looping of CNS0 and CNS2 with the Foxp3 promoter); and administering the regulatory T cell to a subject with cancer.

Examples of cancers that can be treated using the methods and compositions described by the disclosure include, but are not limited to, brain cancer, breast cancer, bladder cancer, pancreatic cancer, prostate cancer, liver cancer, kidney cancer, lymphoma, leukemia, lung cancer, colon cancer ovarian cancer, gastric cancer, cervical cancer, gliomas, head and neck cancers, esophagus cancer, gall bladder cancer, thyroid cancer, and melanoma.

Treg cells used in the treatment of cancer can further be contacted or enhanced with an agent that activates T effector cells. Accordingly, the method may further include administering to the subject an agent that activates T effector cells. In some aspects, an agent that activates T effector cells is an inhibitor of an immune checkpoint protein. Examples of immune checkpoint proteins include inhibitory receptors and their cognate ligands. Examples of inhibitory receptors include, but are not limited to, Cytotoxic T-cell-Lymphocyte-associated Antigen 4 (CTLA4), Programmed Cell Death protein 1 (PD1), Lymphocyte Activation Gene 3 (LAG3), T-cell Membrane Protein 3 (TIM-3), 4-1BB (CD137), and T cell immunoreceptor with Ig and ITIM domains (TIGIT). Examples of immune checkpoint proteins that are ligands include, but are not limited to, PD1 Ligands 1 and 2 (PDL-1, PDL-2), B7-H3, B7-H4, and 4-1BB (CD137) ligand. Non-limiting examples of antibody immune checkpoint inhibitors include Ipilimumab, Tremelimumab, MDX-1106 (BMS-936558), MK3475, CT-011 (Pidilizumab), MDX-1105, MPDL3280A, MEDI4736, and MGA271.

In some aspects, the method of treating cancer further includes administering to the subject a chemotherapeutic agent. Generally, chemotherapeutic agents are classified by their mode of action and/or chemical structure. Examples of chemotherapeutic drug classes include alkylating agents (e.g., nitrogen mustards such as cyclophosphamide, nitrosureas, alkyl sulfonates, triazines, ethlyenimines), platinum drugs (e.g., cisplatin, carboplatin, oxalaplatin), antimetabolites (e.g., 5-fluorouracil (5-FU), 6-mercaptopurine (6-MP), capecitabine, cytarabine, floxuridine, fludarabine, gemcitabine, hydroxyurea, methotrexate, pemetrexed), anti-tumor antibiotics (e.g., daunorubicin, doxorubicin, epirubicin, idarubicin, actinomycin-D, bleomycin, mitomycin-C, mitoxantrone), topoisomerase inhibitors (e.g., topotecan, irinotecan (CPT-11), etoposide (VP-16), teniposide), mitotic inhibitors (e.g., paclitaxel, docetaxel, ixabepilone, vinblastine, vincristine, vinorelbine, estramustine), corticosteroids (e.g., prednisone, methylprednisone, dexamethasone), and monoclonal antibodies (e.g., gemtuzumab, brentuximab, trastuzumab, bevacizumab, cetuximab, rituximab).

In some cases, such as autoimmune diseases, metabolic dysregulation, tissue regeneration, allograft transplantation, and age-related neurodegeneration, it is desirable to inhibit or suppress the differentiation of Treg cells. For example, in the context of autoimmune disease, it may be beneficial to stabilize suppressive Treg phenotypes by preventing differentiation of Treg cells into T effector cells. Therefore, in some aspects, this invention provides a method for treating an autoimmune disease or condition by increasing the activity of conserved CNS0 and CNS2 in a regulatory T cell; and administering the regulatory T cell to a subject with an autoimmune disease or condition, thereby treating the subject's autoimmune disease or condition.

Examples of autoimmune diseases that can be treated with the methods and compositions described herein include Addison's disease, ankylosing spondylitis, alopecia areata, autoimmune hemolytic anemia, autoimmune hepatitis, dermatitis, dermatomyositis, diabetes (type 1), juvenile idiopathic arthritis, glomerulonephritis, Goodpasture's syndrome, Graves' disease, Guillain-Barrë syndrome, Hashimoto's thyroiditis, hemolytic anemia, idiopathic thrombocytopenic purpura, Henoch-Schonlein purpura (HSP), juvenile arthritis, juvenile myositis, Kawasaki disease, inflammatory bowel diseases (such as Crohn's disease and ulcerative colitis), myasthenia gravis, myocarditis, multiple sclerosis (MS), neuromyelitis optica, pemphigus/pemphigoid, pernicious anemia, polyarteritis nodosa, polymyositis, primary biliary cirrhosis, psoriasis, psoriatic arthritis, pulmonary alveolar proteinosis, rheumatoid arthritis (RA), scleroderma/systemic sclerosis, Sjögren's syndrome (SjS), systemic lupus erythematosus (SLE), thrombocytopenic purpura, thyroiditis, uveitis, vasculitis, vitiligo, and granulomatosis with polyangiitis (Wegener's granulomatosis).

The invention is further based, at least in part, on the discovery that the CNS0 and CNS3 coordinate to synergistically regulate Treg cell induction from Treg cell precursors. Accordingly, this invention provides a method for treating diseases or conditions caused by impaired Treg cell diversity or coverage by increasing the activity of CNS0 and CNS3 in a Treg cell precursor. Not wishing to be bound by theory, enhanced Treg cell differentiation potential of these precursor cells will generate a more diverse Treg repertoire recognizing self-antigens, food antigens, or commensal bacterial antigens to modulate tissue immune environment and mitigate disease pathology. Subjects benefiting from treatment with modified Treg cell precursors include those having or at risk of having an undesired inflammatory condition such as any one of the autoimmune diseases described herein.

In some embodiments, the Treg cell and Treg cell precursors express an antigen-binding receptor (e.g., T cell antigen receptor (TCR) or chimeric antigen receptor (CAR)) targeting an autoantigen associated with an autoimmune disease, such as myelin oligodendrocyte glycoprotein (multiple sclerosis), myelin protein zero (autoimmune peripheral neuropathy), myelin basic protein (multiple sclerosis), CD37 (systemic lupus erythematosus), CD20 (B-cell mediated autoimmune diseases), and IL-23R (inflammatory bowel diseases such as Crohn's disease or ulcerative colitis).

The Treg cell and Treg cell precursors of the present invention can be used in cell therapy to treat a patient (e.g., a human patient) in need of Treg cell suppression or induction of immune tolerance or restoration of immune homeostasis. The terms “treating” and “treatment” refer to alleviation or elimination of one or more symptoms of the treated condition, prevention of the occurrence or reoccurrence of the symptoms, reversal or remediation of tissue damage, and/or slowing of disease progression.

A patient herein may be one in need an allogeneic transplant, such as an allogeneic tissue or solid organ transplant or an allogeneic cell therapy. The cells of the present disclosure, such as those expressing CARs targeting one or more allogeneic MHC class I or II molecules, may be introduced to the patient, where the cells will home to the transplant and suppress allograft rejection elicited by the host immune system and/or graft-versus-host rejection. Patient in need of a tissue or organ transplant or an allogeneic cell therapy include those in need of, for example, kidney transplant, heart transplant, liver transplant, pancreas transplant, intestine transplant, vein transplant, bone marrow transplant, and skin graft; those in need of regenerative cell therapy; those in need of gene therapy (AAV-based gene therapy); and those in need of CAR T cell therapy.

Certain aspects of the disclosure include administration of modified Treg cells and Treg cell precursors to a subject; or a composition including the modified Treg cells and Treg cell precursors. In some aspects, a Treg cell is obtained from a subject and modified as described herein to obtain an engineered Treg cell. Thus, in some aspects, an engineered Treg cell is an autologous cell that is administered into the same subject from which an immune cell was obtained. Alternatively, an immune cell is obtained from a subject and is transformed, e.g., transduced, as described herein, to obtain an engineered Treg cell that is allogenically transferred into another subject.

In some aspects, additional steps can be performed prior to administration of a modified Treg cell and Treg cell precursor to a subject. For instance, a modified Treg cell and Treg cell precursor can be expanded in vitro after modification, e.g., silencing of CNS0 and CNS2, but prior to the administration to a subject. In vitro expansion can proceed for 1 day or more, e.g., 2 days or more, 3 days or more, 4 days or more, 6 days or more, or 8 days or more, prior to the administration to a subject. Alternatively, or in addition, in vitro expansion can proceed for 21 days or less, e.g., 18 days or less, 16 days or less, 14 days or less, 10 days or less, 7 days or less, or 5 days or less, prior to administration to a subject. For example, in vitro expansion can proceed for 1-7 days, 2-10 days, 3-5 days, or 8-14 days prior to the administration to a subject.

In some aspects, e.g., during in vitro expansion, a modified Treg cell and Treg cell precursor can be stimulated with an antigen (e.g., a TCR antigen). Antigen-specific expansion optionally can be supplemented with expansion under conditions that non-specifically stimulate lymphocyte proliferation such as, for example, anti-CD3 antibody, anti-Tac antibody, anti-CD28 antibody, or phytohemagglutinin (PHA). The expanded Treg cell and Treg cell precursor, can be directly administered into a subject or can be frozen for future use, i.e., for subsequent administrations to a subject.

In certain aspects, a modified Treg cell and Treg cell precursor is administered prior to, substantially simultaneously with, or after the administration of another therapeutic agent. A modified Treg cell and Treg cell precursor described herein can be formed as a composition, e.g., a modified Treg cell and Treg cell precursor and a pharmaceutically acceptable carrier. In certain aspects, a composition is a pharmaceutical composition including at least one modified Treg cell and Treg cell precursor described herein and a pharmaceutically acceptable carrier, diluent, and/or excipient. Pharmaceutically acceptable carriers described herein, for example, vehicles, adjuvants, excipients, and diluents, are well-known and readily available to those skilled in the art. Preferably, the pharmaceutically acceptable carrier is chemically inert to the active agent(s), e.g., a modified Treg cell and Treg cell precursor, and does not elicit any detrimental side effects or toxicity under the conditions of use.

A composition can be formulated for administration by any suitable route, such as, for example, intravenous, intratumoral, intraarterial, intramuscular, intraperitoneal, intrathecal, epidural, and/or subcutaneous administration routes. Preferably, the composition is formulated for a parenteral route of administration.

A composition suitable for parenteral administration can be an aqueous or nonaqueous, isotonic sterile injection solution, which can contain antioxidants, buffers, bacteriostats, and solutes, for example, that render the composition isotonic with the blood of the intended recipient. An aqueous or nonaqueous sterile suspension can contain one or more suspending agents, solubilizers, thickening agents, stabilizers, and preservatives.

Dosage administered to a subject, particularly a human, will vary with the particular method of treatment, the composition employed, the method of administration, and the particular site and subject being treated. However, a dose should be sufficient to provide a therapeutic response. A clinician skilled in the art can determine the therapeutically effective amount of a composition to be administered to a human or other subject in order to treat or prevent a particular medical condition. The precise amount of the composition required to be therapeutically effective will depend upon numerous factors, e.g., such as the specific activity of the modified Treg cells and Treg cell precursors, and the route of administration, in addition to many subject-specific considerations, which are within those of skill in the art.

Any suitable number of modified Treg cells or Treg cell precursors can be administered to a subject. While a single modified Treg cell and Treg cell precursor described herein is capable of expanding and providing a therapeutic benefit, in some embodiments, 10² or more, e.g., 10³ or more, 10⁴ or more, 10⁵ or more, or 10⁸ or more, modified Treg cells or Treg cell precursors are administered. Alternatively, or additionally 10¹² or less, e.g., 10¹¹ or less, 10⁹ or less, 10⁷ or less, or 10⁵ or less, modified Treg cells or Treg cell precursors described herein are administered to a subject. In some embodiments, 10²-10⁵, 10⁴-10⁷, 10³-10⁹, or 10⁵-10¹⁰ modified Treg cells or Treg cell precursors described herein are administered.

A dose of a modified Treg cell or Treg cell precursor described herein can be administered to a subject at one time or in a series of sub-doses administered over a suitable period of time, e.g., on a daily, semi-weekly, weekly, bi-weekly, semi-monthly, bi-monthly, semi-annual, or annual basis, as needed. A dosage unit including an effective amount of a modified Treg cell or Treg cell precursor may be administered in a single daily dose, or the total daily dosage may be administered in two, three, four, or more divided doses administered daily, as needed.

Route of administration can be parenteral, for example, administration by intravenous injection, transpulmonary administration, or transcutaneous administration. Administration can be systemic or local by intravenous injection, intramuscular injection, intraperitoneal injection, or subcutaneous injection.

In addition to treatment, the findings presented herein can be readily applied to the diagnosis of human immunological diseases including cancer where Treg dysregulation plays an important role.

### Example 1: Materials and Methods

Mice. All mice were maintained in the Animal Resources Center at St. Jude Children's Research Hospital under specific pathogen-free conditions, and the experiments were approved by the institutional animal care and use committee. Foxp3^{GFP}, Foxp3^{ΔCNS2-GFP}, Foxp3^{ΔCNS3-GFP}, Foxp3^{ΔCNS0-GFP} and Foxp3^{YFP CRE} on a C57BL/6 background have been previously described (Dikiy et al. (2021) Immunity 54:931-946.e11; Rubtsov et al. (2008) Immunity 28:546-558; Zheng et al. (2010) Nature 463:808-812). Foxp3^{null} mice were also generated and developed lethal autoimmunity as the scurfy mice do (Brunkow et al. (2001) Nat. Genet. 27:68-73; Fontenot et al. (2003) Nat. Immunol. 4:330-336). Rag1^{-/-} C57BL/6 and CD45.1 C57BL/6 mice were purchased from The Jackson Laboratory. Homozygous Rosa^{CAS9} mice constitutively expressing Cas9 (Platt et al. (2014) Cell 159:440-455) were bred with Foxp3^{ΔCNS2-GFP} or Foxp3^{ΔCNS0-GFP} mice to generate Rosa^{CAS9/+} Foxp3^{ΔCNS2-GFP} or Rosa^{CAS9/+} Foxp3^{ΔCNS0-GFP} mice for isolating T cells for CRISPR experiments. Because the Foxp3 gene is located on the X chromosome and male Foxp3^{null} ΔCNS0,2 and ΔCNS0,3 mice developed lethal, early-onset autoimmunity that causes infertility, only male wild-type (WT) GFP, Foxp3^{null}, ΔCNS0, ΔCNS2, ΔCNS3, ΔCNS0,2, and ΔCNS0,3 mice were used in this study. All these mice carry the same GFP-Foxp3 fusion protein engineered at the endogenous Foxp3 locus.

Generation of ΔCNS0,2 and ΔCNS0,3 Mice. ΔCNS0,2 and ΔCNS0,3 mice were generated on a C57BL/6 background by using the CRISPR/Cas9 platform (Qin et al. (2016) Curr. Protoc. Mouse Biol. 6:39-66). Briefly, the pronuclei of Foxp3^{ΔCNS0-GFP} (ΔCNS0) zygotes were injected with several picoliters per reagent mix of 60 ng/μl Cas9 protein (St. Jude Protein Production Core) and 15 ng/μl each of 59 and 39 chemically modified single guide RNA (sgRNA) (Synthego). CNS2 and CNS3 deletion reagents were injected independently. Approximately 25 injected zygotes were transferred into an infundibulum of a 0.5-d postconception pseudopregnant CD-1 foster mother and carried to term. Founder mice were genotyped via targeted high-throughput sequencing on a MISEQ® sequencer (Illumina) and analyzed by using CRIS.py (Connelly & Pruett-Miller (2019) Sci. Rep. 9:4194). After the deletion was confirmed, mice were backcrossed to C57BL/6 mice at least three times. Tissue Lymphocyte Preparation and Flow Cytometry Analysis. Lymphocytes from lymphoid and nonlymphoid organs were prepared according to known methods, with a few modifications (Feng et al. (2014) Cell 158:749-763; Feng et al. (2015) Nature 528:132-136). To isolate lymphocytes from the thymus, spleen, and lymph nodes (LNs), lymphoid organs were dissected after euthanasia, mechanically dissociated, and passed through a 100-μm strainer. To isolate lymphocytes from the liver and lung, mice were first perfused with 1× phosphate-buffered saline (PBS) after euthanasia. Tissues were then cut into small fragments of <1 mm in diameter and digested with 1 mg/ml collagenase IV (Worthington) and 0.25 mg/ml DNase I (Sigma) in DMEM, 10% newborn calf serum (NCS), and 15 mM HEPES at 37° C. for 30 minutes with shaking. Digested samples were passed through a 100-μm strainer, washed once with complete RPMI1640, and then fractionated by centrifugation in 40% silica-based density centrifugation media sold under the tradename PERCOLL® (GE Healthcare) in 1×PBS supplemented with 10% NCS to remove debris. The resulting cell pellets were resuspended in complete RPMI1640. To isolate lymphocytes from small intestine, Peyer's patches were removed, and the remaining tissue was cut into 5-mm fragments and washed with 5% NCS in 1×PBS. Intraepithelial lymphocytes were released by incubating the tissue with 1×PBS, 5% NCS, and 5 mM EDTA at 37° C. for 20 minutes with shaking.

After EDTA was removed, lamina propria lymphocytes were released by digesting the tissue with 125 U/ml or 1 mg/ml collagenase IV and 0.25 mg/ml DNase I in DMEM, 10% NCS, and 15 mM HEPES at 37° C. for 45 minutes with shaking. Digested tissue was then filtered through a 100-μm strainer, pelleted by centrifugation, and fractionated by two layers of silica-based density centrifugation media sold under the tradename PERCOLL® (42% and 70%) in 1×PBS and 5% NCS with centrifugation. The resulting cells between the two layers of silica-based density centrifugation media sold under the tradename PERCOLL® were pelleted and resuspended in complete RPMI1640 with 10% FBS for downstream analyses. All samples were washed by centrifugation in 5-10 ml wash buffer.

For the flow cytometric analysis of cytokine production, lymphocytes were stimulated in vitro with 50 ng/ml phorbol 12-myristate 13-acetate (Sigma) and 0.5 μg/ml ionomycin (Sigma) in the presence of 2 μM monensin (Sigma) at 37° C. for 4 hours. Cells were stained with antibodies against indicated cell surface markers; then, cytokines were stained by using an intracellular staining kit (BD Biosciences).

Cell staining and flow cytometric analyses were performed according to established methods (Feng et al. (2014) Cell 158:749-763; Feng et al. (2015) Nature 528:132-136). Cells were stained with a fixable viability dye, incubated with indicated antibodies against cell surface markers, and fixed/permeabilized with a Foxp3/Transcription Factor Staining Buffer Set (eBioscience) and then stained for intracellular Foxp3, if needed. Cells were fixed in 1% paraformaldehyde for 10 minutes after staining. The following cell-surface proteins were detected with the indicated fluorophore-conjugated antibodies: CD4 (RM4-5; eBioscience), CD8 (5H10; eBioscience), CD25 (PC61.5; eBioscience), CD44 (IM7; eBioscience), CD62L (MEL-14; eBioscience), CTLA-4 (UC10-4B9; eBioscience), KLRG1 (2F1; eBioscience), TCRβ (BioLegend), CD45.1 (A20; eBioscience), and CD45.2 (104; eBioscience). The following intracellular proteins were detected with indicated antibodies: Foxp3 (FJK-16s; eBioscience), Ki67 (B56; BD Biosciences), IL-13 (eBio13A; eBioscience), IL-17 (eBio17B7; eBioscience), IFN-γ (XMG1.2; eBioscience), and IL-2 (JES6-5H4; eBioscience). To track cell division, cells were labeled with cell-permeant carboxyfluorescein succinimidyl ester sold under the tradename CELLTRACE® CFSE (Thermo Fisher Scientific) according to the manufacturer's instructions.

FACS analyses were performed on LSR II or LSRFortessa (BD Biosciences) flow cytometers. Data were analyzed via FlowJo software (BD Biosciences).

T Cell Sorting and Culture. CD4 T cells were enriched by using EasySep™ Mouse CD4 T Cell Isolation Kits (STEMCELL) to process cells from lymph nodes (LNs) and spleens. CD4 naive T (Tn) cells (CD4⁺CD25⁻CD44^{lo}CD62L^{hi}) and natural Treg (nTreg) cells (CD4⁺GFP⁺) were further FACS sorted from Foxp3^{GFP}, ΔCNS0, ΔCNS2, ΔCNS3, ΔCNS0,2, or ΔCNS0,3 mice. CD4 Tn and nTreg cells were isolated from ΔCNS2 Rosa^{CAS9/+} or ΔCNS0 Rosa^{CAS9/+} mice for CRISPR experiments. nTreg cells were double sorted to ensure >99% purity. T cells were cultured at 37° C., 5% CO₂ in RPMI1640 supplemented with 10% FBS, 2 mM L-glutamine, 1 mM sodium pyruvate, 1% nonessential amino acid, 10 mM HEPES, 20 μM 2-mercaptoethanol, 100 U/ml penicillin, 100 mg/ml streptomycin (complete RPMI1640), and indicated cytokines and compounds.

Treg Cell Induction and Stability Assay. Induction of Treg cell differentiation in vitro was conducted according to established protocols (Feng et al. (2015) Nature 528:132-136; Yue et al. (2016) J. Exp. Med. 213:377-397). Briefly, cell culture plates or dishes were precoated with 1 μg/ml anti-CD3 (Bio X Cell) and anti-CD28 (Bio X Cell) antibodies in PBS at 37° C. for 2 hours and washed with PBS once before cell culture. CD4 Tn cells were grown in complete RPMI1640 supplemented with 100 U/ml recombinant human IL-2 (PeproTech) and 1 ng/ml recombinant human TGF-β (R&D Systems) with or without 0.25 mM ascorbic acid (ASC)-2-phosphate (Sigma) for 4 days.

To assess the stability of Foxp3 expression in vitro, in vitro Treg (iTreg) cells, sorted based on GFP-Foxp3 expression after 4 days of induction, were further cultured on plates coated with both anti-CD3 (1 μg/ml) and anti-CD28 (1 μg/ml) antibodies and either recombinant human IL-2 or 25 μg/ml IL-2 neutralization antibodies (Bio X Cell) for 4 days more. To culture nTreg cells, FACS-sorted nTreg cells were cultured in complete RPMI1640 supplemented with 10% FBS in the presence of DYNABEADS® Mouse T-Activator CD3/CD28 Beads (Thermo Fisher Scientific) with 500 U/ml recombinant human IL-2.

Assessment of Treg Cell Lineage Stability in Lymphopenic Mice. Activated Treg cells)(CD4⁺GFP⁺CD44^{hi}CD62L^{lo}) and resting Treg cells (CD4⁺GFP⁺CD44^{lo}CD62L^{hi}) were sorted from the spleen and LNs of CD45.1⁻CD45.2⁺ Foxp3^{GFP}, ΔCNS0, ΔCNS2, or ΔCNS0,2 mice and mixed at a 1:1 ratio of activated Treg (aTreg) and resting Treg (rTreg) cells. To assess the stability of ΔCNS0,3 nTreg cells, CD4⁺GFP⁺ nTreg cells were sorted from the spleens and LNs of CD45.1⁻CD45.2⁺Foxp3^{GFP}, ΔCNS0, ΔCNS3, ΔCNS0,3 mice. Then, 100,000 CD45.1⁻CD45.2⁺ Treg cells were cotransferred into each Rag1^{-/-} recipient mouse via intravenous injection with 1 million CD4 Tn cells and 1 million CD4 T cells isolated from WT CD45.1⁺CD45.2⁻ or CD45.1⁺CD45.2⁺ male mice. The lymphocyte subsets were analyzed by flow cytometry 3-4 weeks later.

CRISPR-Tiling Library Construction. A retroviral sgRNA library was designed targeting all the sites bearing protospacer-adjacent motifs (PAMs) throughout a 20-kb region surrounding the Foxp3 locus (from 12.5 kb upstream to 7.5 kb downstream of the transcription start site). Specifically, all PAM sequences (NGG) were first identified within the X chromosome: 7143000-7165000 (mm9). To ensure the target specificity, the targets with low complexity sequences, such as repetitive ACG and TTTT, were filtered. The targets ending with PAM and bearing unique sequence specificity in the mouse genome were then selected. In addition, 100 nontargeting negative controls were added to the library. A library containing 75-nt single-stranded DNA oligonucleotides was synthesized by using a high-throughput method (GenScript) and amplified by PCR with primers binding to the flanking arms according to standard protocols. PCR product was then assembled with BbsI-linearized pSIR-BbsI-Thy1.1 vector backbone (modified from pSIR-hCD2; Addgene) using a Gibson Assembly Master Mix (New England Biolabs). The resulting product was electroporated into Escherichia coli (New England Biolabs) and selected by ampicillin on 16 25-cm² dishes. Transformation efficiency was quantified to ensure sufficient coverage. Plasmid was extracted by using the COMPACTPREP® Plasmid Maxi Kit (QIAGEN), and the sgRNA coverage was validated by performing high-throughput sequencing.

Retroviral Packaging and Transfection. Packaging of retrovirus and transduction of mouse primary T cells were conducted according to known protocols, with a few modifications (Feng et al. (2015) Nature 528:132-136). Specifically, platinum-E cells were used for packaging the retrovirus per the manufacturer's instructions (Cell Biolabs). Cells were grown in DMEM supplemented with 10% FBS, 10 mM HEPES, 1% nonessential amino acid, 100 U/ml penicillin, and 100 mg/ml streptomycin. To maintain the packaging vectors, cells were cultured in 1 μg/ml puromycin and 10 μg/ml blasticidin. One day before transfection, cells were seeded on new dishes or plates in medium without puromycin or blasticidin. Then, sgRNA-expressing constructs and pCL-Eco plasmid (Addgene) were cotransfected into these cells with TRANSIT®-293 Transfection Reagent (Mirus). Approximately 18 hours after transfection, cells were given fresh complete DMEM. Virus-containing medium was collected 48 hours and 72 hours after transfection, aliquoted, and stored at −80° C.

Retrovirus Transduction. The titers of retroviral preparations were quantified before experiments to achieve expected transduction efficiency. To this end, 4×10⁴ CD4 Tn cells were seeded in one well of 96-well plates coated with 1 μg/ml anti-CD3 antibodies and 1 μg/ml anti-CD28 antibodies in Treg cell-inducing conditions (i.e., complete RPMI1640 supplemented with recombinant human IL-2 and recombinant human TGF-β). After 72 hours, 10 μg/ml Polybrene (Sigma), 10 mM HEPES, and titrated amounts of viral preparations were mixed and added to the cells. Transduction was performed by centrifuging the cells at 1,200 g at 35° C. for 90 minutes. After transduction, the culture medium was replaced with fresh complete RPMI1640 supplemented with recombinant IL-2 and TGF-β. Eighteen hours after transduction, cells were resuspended, and the culture was split by reseeding onto uncoated plates. Transduction efficiency and viral titers were determined 3 days later by quantifying Thy1.1- or dsRed-expressing cells via flow cytometry.

CRISPR-Tiling Screening for Genetic Elements Controlling Treg Cell Induction. CD4 Tn cells sorted from ΔCNS0 Rosa^{CAS9/+} mice were seeded on culture plates precoated with 1 μg/ml anti-CD3 and 1 μg/ml anti-CD28 antibodies in complete RPMI1640 supplemented with 10% FBS and 100 U/ml recombinant human IL-2. Two days later, cells were transduced with the retroviral library with 10%-20% anticipated efficiency. Transduction was performed as described above in the presence of 6 μg/ml Polybrene and 10 mM HEPES by centrifuging at 1,200 g at 35° C. for 90 minutes. After transduction, cells were cultured in Treg cell-inducing medium (i.e., complete RPMI1640 supplemented with 10% FBS, 100 U/ml recombinant human IL-2, 1 ng/ml recombinant human TGF-β, and 0.25 mM ascorbic acid (ASC)-2-phosphate; Mitsumoto et al. (1994) Biochem. Biophys. Res. Commun. 199:394-402). On day 3, cell cultures were split onto uncoated plates with fresh Treg cell-inducing medium. Cells were harvested on day 6 and stained with Fixable Viability Dye (eBioscience). GFP-Foxp3⁺ and GFP-Foxp3⁻ cells within the live-cell gating were sorted to assess sgRNA representation.

CRISPR-Tiling Screening for Genetic Elements Controlling Treg Cell Lineage Stability. CD4 Tn cells sorted from ΔCNS2 Rosa^{CAS9/+} mice were seeded on cell culture plates precoated with 1 μg/ml anti-CD3 antibodies and 1 μg/ml anti-CD28 antibodies in Treg cell-inducing medium (with 0.25 mM ASC-2-phosphate). Three days later, cells were transduced with the retroviral library with 10%-20% anticipated efficiency. After transduction, the culture medium was replaced with a fresh Treg cell-inducing medium (with 0.25 mM ASC-2-phosphate). Eighteen hours later, cell cultures were split onto uncoated plates with fresh Treg cell-inducing medium (with 0.25 mM ASC-2-phosphate). Cells were harvested on day 6 and stained with Fixable Viability Dye. The top 10% of GFP-Foxp3^{hi} and GFPFoxp3⁻ cells within the live-cell gating were sorted to compare sgRNA representation.

To screen for genetic elements controlling Treg cell stability in ΔCNS0 nTreg cells, nTreg cells double sorted from ΔCNS0 Rosa^{CAS9/+} mice were seeded on 24-well plates at 10⁶ to 2×10⁶ cells per well with Mouse T-Activator CD3/CD28 Beads and 500 U/ml recombinant human IL-2. Cells were transduced with the retroviral library 3 days later, with 10%-20% anticipated efficiency. After transduction, cells were cultured in fresh complete RPMI1640 supplemented with 10% FBS and 500 U/ml recombinant human IL-2. Cells were split onto 12-well plates on day 4 and cultured with fresh Mouse T-Activator CD3/CD28 Beads in complete RPMI1640 supplemented with 500 U/ml recombinant human IL-2 and 10 ng/ml recombinant murine IL-4. Cells were harvested on day 7 and stained with Fixable Viability Dye. The top 10% of GFP-Foxp3^{hi} and GFP-Foxp3⁻ cells within the live-cell gating were sorted for downstream analysis.

sgRNA Recovery. To retrieve sgRNA information, genomic DNA was extracted from sorted T cells by proteinase K digestion, phenol:chloroform: isoamyl extraction, and 2-propanol precipitation. The sgRNA-coding sequences were amplified from genomic DNA by using a two-step PCR protocol. The first PCR (PCR1) amplified the region covering the sgRNA cassette by using primers targeting the retroviral vector backbone and containing the adaptor sequences for the second indexing PCR. The second PCR (PCR2) added barcodes with Illumina i5 and i7 indexing primers. PCR and DNA purification were conducted according to previously published protocols (Yau & Rana (2018) Methods Mol. Biol. 1712:203-216). Specifically, 5 μg genomic DNA was loaded into each 100-μl PCR1 reaction with Q5 Hot Start High-Fidelity DNA Polymerase (New England Biolabs), with the following parameters: 98° C. for 2 minutes, 18 cycles of 98° C. for 20 seconds, 55° C. for 30 seconds, and 60° C. for 45 seconds followed by 65° C. for 5 minutes. Products of PCR1 were purified by using a NUCLEOSPIN® Gel and PCR Clean-up Kit (Macherey-Nagel) and eluted in 100 μl of 10 mM Tris, 1 mM EDTA, pH 8.0, per 100 μl PCR1 reaction. Every 10 μl of eluted PCR1 product was used as a template for a 50-μl PCR2 reaction with the following parameters: 98° C. for 2 minutes, 18 cycles of 98° C. for 20 seconds, 55° C. for 30 seconds, and 65° C. for 45 seconds followed by 65° C. for 5 minutes. Products of PCR2 were separated by electrophoresis through a 1.5% agarose gel, and DNA bands of 261 bp were excised and retrieved by using a NUCLEOSPIN® Gel and PCR Clean-up Kit. Purified PCR2 products were then sequenced by using MISEQ® kits (Illumina) with single-end sequencing for 100 cycles. More than 10⁶ reads were obtained for each sample.

Validation of CRISPR-Tiling Screening Results. To validate the effects of sgCNS1-1, sgCNS3-1, sgCNS3-2 on iTreg cell induction, sgNC was used as a nontargeting negative control and sgCNS2-1 as a neutral reference based on the Treg cell induction screening result. CD4 Tn cells sorted from WT GFP Rosa^{CAS9/+} and ΔCNS0 Rosa^{CAS9/+} mice were transduced with individual sgRNAs and treated in the same way as in the iTreg cell induction CRISPR-tiling screening.

To validate the effects of sgCNS0-1, sgCNS0-2, sgCNS0-3, and sgCNS0-4 on the stability of ASC-pretreated iTreg cells, sgNC was used as a nontargeting negative control and sgCNS1-1 and sgCNS3-3 as neutral references based on the Treg cell stability screening result. CD4 Tn cells sorted from WT GFP Rosa^{CAS9/+} and ΔCNS2 Rosa^{CAS9/+} mice were transduced with individual sgRNAs and treated in the same way as in iTreg cell stability CRISPR-tiling screening. To validate their effects on the stability of nTreg cells, nTreg cells double sorted from WT GFP Rosa^{CAS9/+} and ΔCNS2 Rosa^{CAS9/+} mice were transduced with individual sgRNAs and treated in the same way as in nTreg cell stability CRISPR-tiling screening.

The percentages of GFP-Foxp3⁺ among the live, transduced cells (dsRed⁺) were quantified and normalized to those of sgNC-transduced cells of the same genotypes. The ratios of normalized percentages of GFP-Foxp3⁺ of ΔCNS0 or ΔCNS2 cells to those of WT GFP cells were further calculated.

Generation of mixed bone marrow chimeras Mixed bone marrow chimeras were generated as previously described (Feng et al. (2014) Cell 158:749-763). Briefly, recipient mice (CD45.1⁺CD45.2⁺) were irradiated (950 Rad) 24 hours before intravenous injection of 10×10⁶ bone marrow cells from CD45.1⁺CD45.2⁺ WT codonor mice and CD45.1⁻CD45.2⁺ WT GFP, ΔCNS0, ΔCNS2, ΔCNS3, ΔCNS0,2, or ΔCNS0,3 mice mixed at a 1:1 ratio. After bone marrow transfer, the recipient mice were administered with 2 mg/ml neomycin in drinking water for 3 weeks. Mice were analyzed 8-10 weeks later.

Treg Cell In Vitro Suppression Assay. nTreg cells were FACS sorted from pooled spleens and LNs of 3- to 4-week-old WT GFP and ΔCNS0,3 male littermates or mixed bone marrow chimeric mice of WT GFP and ΔCNS0,3. CD4 Tn cells were sorted from the spleens of CD45.1 mice and stained with CELLTRACE® CFSE according to the manufacturer's protocol (Thermo Fisher Scientific). Antigen-presenting cells were prepared from the splenocytes of male CD45.1 mice by depleting CD90.2⁺ T cells and TER-119⁺ red blood cells followed by lethal irradiation (20 Gy). Treg cell suppression assay was conducted in 96-well U-bottom plates, each well containing 200 μl complete RPMI1640 supplemented with 1 μg/ml anti-CD3 antibody, 10⁵ antigen-presenting cells, and 4×10⁴ CD4 Tn cells. Treg cells were added at different ratios to CD4 Tn cells. Cells were harvested 3 days later for flow cytometric analysis after being stained with viability dye (Tonbo Biosciences) and antibodies against CD4 and CD45.1.

scRNA-seq and TCR V(D)J Clonotype Profiling. To profile differential gene expression of innate and adaptive immune cells, spleens or lungs from two Foxp3^{GFP} and four ΔCNS0,2 mice were pooled. Spleens were ground mechanically, and lungs were digested with 1 mg/ml collagenase IV and 0.25 mg/ml DNase I as described above. The resulting cells were stained with viability dye followed by surface markers. Approximately 90% of B220⁺ cells were depleted. The remaining live single cells were FACS sorted for 10× scRNA-seq. To profile gene expression and TCR V(D)J clonotypes of Treg cells, CD4⁺GFP⁺ cells were sorted from pooled spleens and LNs of mixed bone marrow chimeric mice of WT GFP (n=5), ΔCNS0 (n=5), ΔCNS2 (n=5), and ΔCNS0,2 (n=22, because of the lower frequencies of Treg cells). Cell density and viability of sorted cells were further determined by trypan blue staining and hemocytometer. All the processed samples had cell viability >85%. Sample processing for single-cell gene expression and TCR V(D)J clonotypes was conducted by using the Chromium Single Cell 5′ Library and Gel Bead Kit (10× Genomics) according to the manufacturer's protocol, with an anticipated recovery of 10,000 cells per sample. Altogether, 13 cycles of PCR were used to amplify the cDNA library to produce sufficient material to prepare RNA and TCR V(D)J libraries. TCR V(D)J enrichment was conducted according to the manufacturer's protocols for mouse T cell Chromium Single Cell V(D)J Enrichment Kit (10× Genomics). RNA- and V(D)J-seq libraries were prepared by following the manufacturer's protocol (10× Genomics) and profiled by TAPESTATION® (Agilent Technologies). Sequencing was performed on a NOVASEQ® 6000 sequencer (Illumina) with an expected 500 million clusters for each scRNA-seq library and 50 million clusters for each scTCR V(D)J library, according to the manufacturer's specifications (10× Genomics).

Capture-C. Capture-C experiments were performed as previously described (Davies et al. (2016) Nat. Methods. 13:74-80), with minor modifications (Zhang et al. (2019) Proc. Natl. Acad. Sci. USA 116:26644-26652). In brief, 10⁷ mouse primary T cells were fixed in 1% formaldehyde for 10 minutes at room temperature with gentle rotation. The reaction was quenched by 0.125 M glycine. Cells were then resuspended in 5 ml lysis buffer and incubated on ice for 20 minutes. Cells were pelleted and resuspended in 1 ml of 1×DpnII digestion buffer and transferred to a 2-ml tube for homogenization followed by centrifugation at 14,000 rpm for 5 minutes at 4° C. Complete homogenization was confirmed under the microscope, and the supernatant was removed. The remaining pellets were subjected to DpnII (New England Biolabs) digestion, T4 DNA ligation (Thermo Fisher Scientific), and decrosslinking. The resulting 3C library DNA was precipitated, and a small portion was separated by electrophoresis with an agarose gel to assess the digestion and ligation efficiency. Then, 5 μg of the 3C DNA library was sheared to ˜200 bp followed by end repair, adaptor ligation, and PCR enrichment of adaptor-ligated DNA. Two rounds of captures were conducted at 47° C. by using 2 μg of DNA, 5 μg mouse Cot-1 DNA (Thermo Fisher Scientific), biotinylated DNA oligonucleotide probes targeting the Foxp3 promoter region, 1 μl xGen Universal Blocking Oligo i7, and 1 μl xGen Universal Blocking Oligo i5 (Integrated DNA Technologies) in a 1.5-ml tube. The constructed library was sequenced with paired ends of 150 cycles each.

Paired-end reads from Capture-C were mapped and processed by HiC-Pro (v2.11.4; Servant et al. (2015) Genome Biol. 16:259) to mm9 (MGSCv37 from Sanger) with fragments for DpnII sites. Validated contacts were used to generate BIGWIG files with make_viewpoints.py script (HiC-Pro). Reads were normalized to 100 thousand contacts within 5 million bp of the baitregion. For suitable data visualization, the BIGWIG files were uploaded to the WashU Epigenome Browser and exported as SVG format.

CUT&RUN. nTreg cells (CD4⁺GFP⁺) were sorted from the spleens and LNs of mixed bone marrow chimeric mice. H3K4me3, H3K27ac, and pan-RNA Pol II CUT&RUN was performed according to published protocols, with a few modifications (Skene & Henikoff (2017) eLife 6:e21856). Briefly, 0.2×10⁶ to 0.5×10⁶ Treg cells were attached to concanavalin A-coated magnetic beads (Bangs Laboratories) for each experiment. Cells were permeabilized with digitonin (Sigma) in a wash buffer (20 mM HEPES, pH 7.5, 150 mM NaCl, 0.5 mM spermidine, 0.01% digitonin, and protease inhibitors) and then incubated with primary antibodies against H3K4me3 (Active Motif), H3K27ac (Abcam), or pan-Pol II (Abcam) at 4° C. for 2 hours on a rotator. The beads-cells mixture was washed three times with digitonin wash buffer, resuspended in 200 μl protein A-MNase (Addgene), and incubated at 4° C. for 1 hour on a rotator. After three rounds of washing, beads were resuspended in 150 μl digitonin wash buffer and chilled to 0° C. for 5 minutes. Then, 3 μl of 100 mM CaCl₂ was added into the tubes with gentle vortexing, and the tubes were placed in an ice-water bath. After 30 minutes of incubation, 150 μl of 2× STOP buffer (170 mM NaCl, 20 mM EDTA, 20 mM EGTA, 0.05% digitonin, 20 mg/ml GLYCOBLUE™, and 25 mg/ml RNase A) was added. Beads were incubated at 37° C. for 30 minutes and then placed on a magnet stand for 2 minutes. The clarified liquid was transferred to a new tube for phenol:chloroform extraction followed by DNA precipitation by ethanol. The sequencing library was prepared by using KAPA HyperPrep Kit (Kapa Biosystems) according to the manufacturer's instructions.

Whole-Genome Bisulfite Sequencing (WGBS). nTreg cells (CD4⁺GFP⁺) were sorted from the spleens and LNs of mixed bone marrow chimeric WT GFP, ΔCNS0, ΔCNS2, or ΔCNS0,2 mice. CD4 Tn, T_{E}, and Treg cells were sorted from male WT GFP mice (Tn cells: CD4⁺GFP⁻CD25⁻CD44^{lo}CD62L^{hi}; T_{E} cells: CD4⁺GFP⁻CD44^{hi}CD62L^{lo}; Treg cells: CD4⁺GFP⁺). iTreg and ASC-treated iTreg cells were induced from CD4 Tn cells of male WT GFP mice in vitro with or without ASC (as described above), and GFP⁺ Treg cells were sorted on day 4 of in vitro culture. Genomic DNA was prepared from sorted cells via proteinase K digestion followed by phenol:chloroform:isoamyl alcohol extraction and 2-propanol precipitation. More than 100 ng of genomic DNA in each sample was converted by using EPITECT® Bisulfite Kits (QIAGEN). Libraries were prepared from converted DNA by using the ACCEL-NGS® Methyl-Seq DNA Library Kit (Swift Biosciences). Libraries were analyzed for insert-size distribution on a 2100 BioAnalyzer after processing using a High Sensitivity DBA Kit (Agilent Technologies), on an Agilent 4200 TAPESTATION® system after processing using a D1000 SCREENTAPE® assay, or on a Caliper LABCHIP® GX microfluidic instrument after processing using a DNA High Sensitivity Reagent Kit (Perkin-Elmer) and then quantified by using the Quant-iT™ PICOGREEN® dsDNA assay (Life Technologies) or low-pass sequencing with a MISEQ® Nano Kit (Illumina). Paired-end, 150-cycle sequencing was performed on a NOVASEQ® 6000 sequencer (Illumina) to achieve an average 40× coverage.

ATAC-seq. ATAC-seq was performed as previously reported (Buenrostro et al. (2013) Nat. Methods 10:1213-1218), with a few modifications. Briefly, 5×10⁴ cells were FACS sorted, washed once with ice-cold PBS, and lysed in 300 μl lysis buffer (10 mM Tris-HCl, pH 7.5, 10 mM NaCl, 3 mM MgCl₂, and 0.1% NP-40) by gently pipetting up and down. After centrifugation, the supernatant was removed. Then, 50 μl of reaction mix containing 25 μl TD Buffer, 2.5 μl TDE1 (Illumina Nextera DNA Library Prep Kit), and 22.5 μl nuclease-free water was immediately added to set up a transposition reaction at 42° C. for 40 minutes. DNA was immediately purified afterward using the NUCLEOSPIN® Gel and PCR Clean-up Kit (Macherey-Nagel). The transposed DNA was amplified by PCR for 10-12 cycles with the NEXTERA® DNA Library Prep Kit and NEXTERA® XT Indexing Kit (Illumina). The library DNA within the 150-500-bp range was enriched by one round of negative selection with 0.6 volume AMPURE® XP beads (Beckman Coulter) and two rounds of positive selection with 1 volume AMPURE® XP beads. The libraries were quantified by NEBNEXT® Library Quant Kit for Illumina (New England Biolabs) and sequenced, with paired-end 100-cycle sequencing performed on a HISEQ® 4000 or HISEQ® 2500 sequencer (Illumina).

Bulk TCR Repertoire Profiling. Bulk TCR sequencing was performed as previously described (Dash et al. (2015) Methods Mol. Biol. 1343:181-197; Egorov et al. (2015) J. Immunol. 194:6155-6163; Feng et al. (2015) Nature 528:132-136). Briefly, CD4⁺GFP-Foxp3⁺ nTreg cells were sorted from the spleen, skin draining LN, and mesenteric LN (mLN) of individual WT GFP, ΔCNS0, ΔCNS3, and ΔCNS0,3 mice at 16 days of age. Total RNA was extracted by using TRIZOL® Reagent (Life Technologies). All the RNA was used for cDNA synthesis and TCR sequencing library preparation. cDNA was synthesized by SMARTScribe reverse transcription (Takara), with primers targeting the constant regions of mouse TCRα (5′-AGTCAAAGTCGGTGAAC-3′, SEQ ID NO:4) or TCRβ (5′-ATCTCTGCTTTTGATG-3′, SEQ ID NO:5) chains and template switch adapter integrated with unique molecular identifiers (5′-AAG CAGUGGTAUCAACGCAGAGUNNNNUNNNNUNNNNUCTTrGrGrGrG-3′, SEQ ID NO:6). The TCRα and TCRβ chains were amplified with a two-step protocol: First, a mixture of primers (5′-CACTCTATC CGACAAGCAGTGGTATCAACGCAG-3′, SEQ ID NO:7; and 5′-CACTCTATCCGA CAAGCAGT-3′, SEQ ID NO:8) bound at the template switch adapter and primers targeting the constant regions of TCRα or TCRβ chains (TCRα: 5′-GCTGTCCTGAGACCGAGGAT-3′, SEQ ID NO:9; TCRβ: 5′-ATGGCTCAAACAAGGAGACC-3′, SEQ ID NO:10) were used. Second, indexing primer 5′-(N)₂₋₄(XXXXX)CAGTGGTATCAACGCAGAG-3′ (SEQ ID NO:11) annealing on the template switch adapter was used together with TCRα 39 indexing primer 5′-(N)₂-4(XXXXX)CAGGTTCTGGGTTCTGGATGT-3′ (SEQ ID NO:12) or TCR 3′ indexing primer 5′-(N)₂₋₄ (XXXXX)AGTCACATTTCTCAGATCCT-3′ (SEQ ID NO:13). Sequencing adapters were then added using the KAPA HyperPrep Kit. Samples were pooled and sequenced on a NOVASEQ® 6000 sequencer (Illumina) with paired-end reads of 150 cycles each. Reads with at least 10× coverage relative to the input cell numbers were acquired. Note: (N)₂₋₄ indicates two to four random nucleotides for efficient separation of clusters during sequencing; U, uracil for degradation of template switch adaptor by uracil-DNA glycosylase (New England Biolabs) after reverse transcription; rG, riboguanosine for template switch during reverse transcription; and XXXXX, 5-nt barcodes for multiplexing samples.

Histological Analysis. Tissue samples were fixed in 10% neutral-buffered formalin and processed for H&E staining. Stained slides were scored to indicate the grades of tissue inflammation.

Data Analysis: CRISPR-Tiling Screening. To quantify the representation of sgRNAs, the raw FASTQ data were first de-barcoded and mapped to the original reference sgRNA library. The differentially enriched sgRNAs were defined by comparing normalized counts between sorted GFP⁺ and GFP⁻ cells. Normalized counts for each sgRNA were extracted and used to identify differentially enriched sgRNA by DESeq2 (Love et al. (2014) Genome Biol. 15:550).

Data Analysis: Processing, Dimensionality Reduction, and Clustering of 10× Data. More than 730 and 260 million reads were acquired for each scRNA-seq library and scTCR V(D)J library, respectively. Single-cell gene expression was obtained by using 5′ kits from 10× Genomics (v1.1). Libraries were sequenced on the Illumina NOVASEQ® platform at 100×100 bp to obtain at least 50,000 reads per cell on average. For a subset of samples, TCR libraries were also generated from the barcoded single-cell cDNA by using the mouse V(D)J enrichment kit (10× Genomics) and sequenced at 150×150 bp to obtain at least 5,000 reads per cell on average. Read processing, read alignment, cell-barcode demultiplexing, and gene-specific unique molecular identifier (UMI) counting was performed by using CellRanger (v3.1.0) and the corresponding mouse references (mm10 v3.0.0 for gene expression, vdj-GRCm38-alts-ensembl-3.1.0 for TCR). Gene expression data obtained from distinct samples within the same experiment were subsequently aggregated by CellRanger and normalized by read depth across libraries by subsampling to obtain a similar average number of mapped reads per cell across cells. The average number of reads per cell after normalization was 92,531 for assessment of immune cell activation in WT GFP and ΔCNS0,2 mice with scRNA-seq and 66,111 when assessing the functional states of ΔCNS0,2 Treg cells.

Downstream analyses of filtered feature barcode matrices were conducted separately for each experiment using Seurat (Stuart et al. (2019) Cell 177:1888-1902.e21). Genes observed in <0.1%-0.2% of cells were excluded from downstream analysis. Cells that exhibited extremes in the percentage of mitochondrial expression, number of genes expressed, or number of transcripts expressed were excluded. Data were normalized by using default parameters, and the top 2,000 variable genes were detected by using the ‘vst’ method after, excluding clonotype-specific V(D)J gene segments. The cell-cycle phase was inferred using known markers (Tirosh et al. (2016) Science 352:189-196). Variable genes were then scaled and centered by their variation across the dataset while regressing out the effects of the number of UMIs, percentage of mitochondrial gene expression, and the cell-cycle phase scores per cell. Principal component (PC) analysis was then conducted on the scaled genes, wherein the PCs to use for downstream dimensionality reduction by were selected by either considering the variation accounted for in the PC analysis plot (i.e., assessment of immune cell activation in WT GFP and ΔCNS0,2 mice with scRNA-seq) or the significance of the PCs after random permutation (i.e., assessing the functional states of ΔCNS0,2 Treg cells). These PCs were used to construct shared nearest neighbor graphs, define transcriptional clusters, and generate two-dimensional t-distributed stochastic neighbor embedding plots by using default parameters or uniform manifold approximation and projection plots.

Data Analysis: Cell-Type Identification in 10× Data. Seurat (Stuart et al. (2019) Cell 177:1888-1902.e21) was applied for scRNA-seq analysis and data visualization. High mitochondrial RNA (>8%) and low-UMI cells (<400) were removed from the single-cell dataset. Cellular identities were determined by the marker genes found by the Seurat R package. Erythroid cells (Hba-a1⁺ and Hba-a2⁺), endothelial cells (Pecam1⁺ and Epcam⁺), and fibroblasts (Col3a1⁺ and Colla1⁺) were excluded because these cells were CD45⁻ cells which were irrelevant to the studies herein. Further cell identification was based on gene expression of known cell markers for each cluster: Cd3e, Cd4, CD8a, Foxp3, Sell, Ccr7, Gzma, Gzmb, Cd44, Cd16311, Gata3, Ncr1, Klrb1c, Igta2, Klra7, Zbtb16, Ly6g, Ly6c1, Itgax, Itgam, Cd63, Cd200r3, Lyz2, Cd14, Adgre1, and Fcgr4. Based upon this analysis, 4,199 cells from spleens of WT GFP mice, 3,696 cells from the spleens of zCNS0,2 mice, 4,639 cells from lungs of WT GFP mice, and 5,546 from lungs of ΔCNS0,2 mice were obtained. Violin plots were constructed with the Seurat package's implementation function VlnPlot( ) to show individual gene expression in different cell subsets.

Data Analysis: Analysis of Differential Gene Expression of 10× Data. The FindMarkers function from Seurat (Stuart et al. (2019) Cell 177:1888-1902.e21) was used to identify the differentially expressed genes for each cluster compared with other clusters. The top 20 significant genes (min.pct=0.001, logfc.threshold=0.1) ordered by false discovery rate-corrected P values (<0.01) were extracted to plot the heatmaps (DoHeatmap function). Low-confidence clusters C15 and C16 were excluded because not all genotypes had >50 cells.

Data Analysis: Pseudotime Modeling of Treg Cell Heterogeneity. Processed results from Seurat were first imported into monocle (v2.9.0; Qiu et al. (2017) Nat. Methods 14:979-982). Based on the elbow of variance-explained plot (plot_pc_variance explained function), 10 PCs were chosen to construct the spanning tree (max_components=2; num_dim=10) with DDRTree (v0.1.5). Cell states assigned by monocle were extracted for comparison among genotypes. Cell trajectories were plotted by monocle and colored by pseudotime (plot cell trajectory function).

Data Analysis: TCR Clonotype Analysis of 10× Data. TCR or V(D)J sequences were extracted by CellRanger (v3.1.0). TCR clones were counted across genotypes. To visualize Treg cell activation states and associated TCR copy numbers, TCR clonotypes were overlaid with Seurat clusters by using Loupe Browser (10× Genomics), and TCRs with different copy numbers were highlighted.

Data Analysis: Analysis of Bisulfite Sequencing Data. WGBS data were aligned to mouse genome mm9 assembly using BSMAP2.74 (Xi & Li (2009) BMC Bioinformatics 10:232). The methylation ratio for each CpG site was extracted by methratio.py from BSMAP2.74. The methylation ratio was then converted to a BW file for visualization. Regions covered by fewer than five reads were marked as −0.2.

Data Analysis: ATAC-Seq. Paired-end reads of 100 bp were trimmed by Cutadapt (v1.9, paired-end mode, default parameter with “-m 25-O 6”; Martin (2011) EMBnet. J. 17:10-12) and aligned to mouse genome mm9 (MGSCv37 from Sanger) by BWA (v0.7.12-r1039, default parameter; Li & Durbin (2009) Bioinformatics 25:1754-1760). Duplicate reads were marked with biobambam2 (v2.0.87; Tischler & Leonard (2014) Source Code Biol. Med. 9:13); only nonduplicate, properly paired reads were kept by the samtools (parameter “-q 1 -F 1804” v1.2; Li et al. (2009) Bioinformatics 25:2078-2079). After adjusting the Tn5 shift (reads were offset by +4 bp for the sense strand and −5 bp for the antisense strand), fragment size was used to separate reads into nucleosome-free, mononucleosome, dinucleosome, and trinucleosome as previously described (Buenrostro et al. (2013) Nat. Methods 10:1213-1218) and BIGWIG files were generated using the center 80 bp of fragments and scale to 20 million nucleosome-free reads. Acceptable nucleosome-free peaks and patterns of mono-, di-, and tri-nucleosomes on IGV (v2.4.13; Robinson et al. (2011) Nat. Biotechnol. 29:24-26) were observed. Both replicates were subsequently merged to enhance the peak calling on nucleosome-free reads by MACS2 (v2.1.1.20160309 default parameters with “--extsize 200 --nomodel”; Zhang et al. (2008) Genome Biol. 9:R137). All important nucleosome-free regions were considered called if a sample had >15 million nucleosome-free reads after merging. To ensure reproducibility, peaks from different cell types were merged to create a set of reference chromatin-accessible regions. Bedtools (v2.24.0; Quinlan & Hall (2010) Bioinformatics 26:841-842) was then used to count nucleosome-free reads from each sample to overlay with the reference regions. As a confirmation of reproducibility, the Spearman correlation coefficient between replicates was larger than that between samples from different groups. To identify the differentially accessible regions, raw nucleosome-free reads counts were first normalized using trimmed mean of M-values and then applying the empirical Bayes statistics test after linear fitting with the voom package (R 3.23, edgeR 3.12.1, limma 3.26.9; Law et al. (2014) Genome Biol. 15:R29).

Data Analysis: CUT&RUN Sequencing. Paired-end reads of 50 bp were mapped to mouse genome mm9 (MGSCv37 from Sanger) by BWA (v0.7.12-r1039, default settings; Li & Durbin (2009) Bioinformatics 25:1754-1760) after adapters were trimmed by cutadapt (v1.9, paired-end mode, parameter “-m 25-O 6”). Duplicated reads were marked with biobambam2 (v2.0.87; Tischler & Leonard (2014) Source Code Biol. Med. 9:13), and unique reads were kept by samtools (v1.2, parameter “-q 1 -F 1804”; Li et al. (2009) Bioinformatics 25:2078-2079). All samples had >5 million fragments as suggested by the CUT&RUN protocol (Skene & Henikoff (2017) eLife 6:e21856) after replicates were merged. BIGWIG files were generated using the center 80 bp of fragments <2,000 bp and normalized to 10 million fragments. Reproducible reference peaks were compiled for each cell type if the peaks called with stringent cutoff overlapped with the peaks called with relaxed cutoff in the other replicate. Reproducible peaks between cell types were merged to create the reference peak set and counted fragments (fragment size <2,000 bp) were overlapped with these merged reference peaks using bedtools (v2.24.0; Quinlan & Hall (2010) Bioinformatics 26:841-842) for each sample. After trimmed mean of M-values normalization (R 3.4.0, edgeR 3.18.1), voom from R package limma 3.34.9 (Law et al. (2014) Genome Biol. 15:R29) was used to identify differential binding sites.

Data Analysis: Bulk TCR Sequencing. To analyze TCR diversity, bulk TCR sequencing data were demultiplexed using MIGEC v1.2.9 software (Shugay et al. (2014) Nat. Methods 11:653-655). V(D)J gene assignments were calculated and filtered with MIGEC software. VDJtools v1.2.1 software (Shugay et al. (2015) PLOS Comput. Biol. 11:e1004503) was used to filter nonfunctional TCR and decontaminate samples. Immunarch package was used to explore the T cell repertoires and compute TCR diversity.

Statistical Analysis. Statistical tests were performed using GraphPad Prism (GraphPad Software) or the R statistical environment. For data with a small sample size, a robust nonparametric Mann-Whitney test was used; otherwise, an unpaired t test or two-way ANOVA with Tukey's multiple comparisons test was applied. *, P<0.05; **, P<0.01; ***, P<0.001; and ****, P<0.0001.

### Example 2: CRISPR Screening Uncovers Synthetic Interactions of Foxp3 Enhancers

To elucidate the transcriptional mechanisms by which Treg cell repertoire diversity and lineage stability are controlled through Foxp3 expression, the known regulatory circuits governing these processes were examined. They appear to act on several distinct Foxp3 cis-regulatory elements (Josefowicz et al. (2012) Annu. Rev. Immunol. 30:531-564), suggesting cooperative activities of the latter. In particular, enhancer CNS0 functions as an IL-2/Stat5 response element (Akamatsu et al. (2019) Sci. Immunol. 4:eaaw2707; Dikiy et al. (2021) Immunity 54:931-946.e11), and CNS0 deficiency (ΔCNS0) reduces thymic Treg cells by two- to three-fold without causing immune activation.

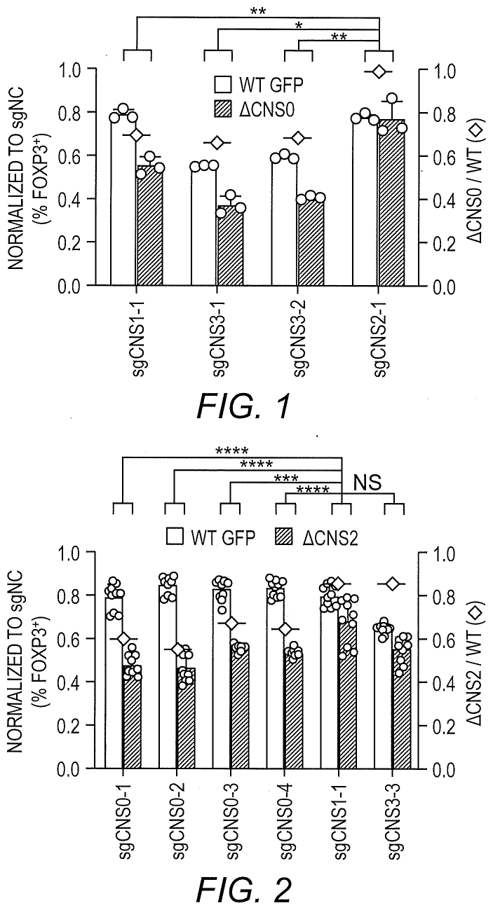

To understand the CNS0-independent mechanisms of Treg cell development that confers immune tolerance in ΔCNS0 mice, a tiling library targeting all the sites bearing the Streptococcus pyogenes Cas9 protospacer-adjacent motifs (PAMs) throughout a 20-kb region surrounding the Foxp3 promoter were constructed to screen for the genetic elements required for in vitro Treg cell (iTreg) development from ΔCNS0 CD4 naïve T cells (Tn) constitutively expressing Cas9. As expected, single guide RNAs (sgRNAs) targeting Foxp3 exons and mRNA splicing sites were highly enriched in Foxp3⁻ cells. Although CRISPR-induced short indels might only partially disrupt enhancers' function (Carleton et al. (2018) J. Vis. Exp. (136):57883), several sgRNAs targeting CNS3 were overrepresented in Foxp3⁻ cells. The additive effects of two of these sgRNAs with ΔCNS0 in iTreg cell induction were verified. As a negative control, the sgRNA targeting CNS2 did not affect Foxp3 induction (FIG. 1). A mild reduction of Foxp3 induction was also observed when CNS1 was targeted by CRISPR in ΔCNS0 cells, suggesting a potential interaction between CNS0 and CNS1. As TCR and IL-2 signaling, two major Treg cell-inducing cues, act through CNS3 (Zheng et al. (2010) Nature 463:808-812) and CNS0 (Dikiy et al. (2021) Immunity 54:931-946.e11), respectively, and CNS0 and CNS3 are modified by enhancer marker H3K4me1 (Feng et al. (2015) Nature 528:132-136; Kitagawa et al. (2017) Nat. Immunol. 18:173-183; Placek et al. (2017) Nat. Immunol. 18:1035-1045; Zheng et al. (2010) Nature 463:808-812) and are bound by Satb1, whose deficiency impairs Treg cell development (Kitagawa et al. (2017) Nat. Immunol. 18:173-183), the results herein further indicate that CNS0 and CNS3 may coordinate to promote Foxp3 induction.

Next, it was determined whether Treg cell lineage stability is controlled by the coordination of Foxp3 enhancers. Because ΔCNS2 partially impairs the stability of Foxp3 expression, CRISPR-tiling screening was performed to search for the genetic elements maintaining Foxp3 expression in ΔCNS2 Treg cells. The same sgRNA library was transduced into ascorbic acid (ASC)-pretreated iTreg cells that recapitulate Treg cell lineage commitment in vivo via Tet-mediated DNA demethylation (Yue et al. (2016) J. Exp. Med. 213:377-397). Several sgRNAs targeting CNS0 (e.g., sgCNS0-1 against a Gata motif) were overrepresented in Foxp3⁻ cells. This result was verified with individual sgRNAs targeting CNS0 in ASC-pretreated iTreg cells and natural Treg cells (nTreg), with sgRNAs targeting CNS1 and CNS3 as negative controls (FIG. 2). CRISPR-tiling screening was also performed with ex vivo-isolated ΔCNS0 nTreg cells and it was found that sgRNAs targeting CNS2, particularly a Stat5 motif, were highly enriched in Foxp3⁻ cells. Thus, CNS0 appears to coordinate with CNS2 to maintain Foxp3 expression, consistent with their Stat5 binding in nTreg cells and the important role of IL-2/Stat5 signaling in maintaining Foxp3 expression (Feng et al. (2014) Cell 158:749-763).

Notably, sgRNAs targeting several nonconserved regions were also enriched or depleted in the CRISPR screening. In addition, there are a few limitations of CRISPR-tiling screening: (1) DNA motifs might not be fully covered by available protospacer adjacent motifs (PAMs), (2) short indels may only partially disrupt enhancer's function, (3) CRISPR-induced DNA breaks might influence Foxp3 transcription by reported mechanisms (Vitelli et al. (2017) Annu. Rev. Genomics Hum. Genet. 18:87-113), and (4) in vitro assays only partially recapitulate Treg cell development and lineage maintenance in vivo. Nonetheless, these results, together with the epigenetic markers and regulatory circuits described above, indicate the coordination of Foxp3 enhancers during Treg cell induction and lineage maintenance.

To understand the mechanistic basis of this coordination, the three-dimensional chromatin architecture around the Foxp3 locus was assessed with Capture-C (Davies et al. (2016) Nat. Methods. 13:74-80; Kim & Dekker (2018) Cold Spring Harb. Protoc. 2018:pdb.top097832) in precursor cells (i.e., CD4 Tn cells), ASC-treated iTreg cells, and nTreg cells. Reproducible, abundant loops were observed between the Foxp3 promoter and enhancers as well as other novel chromatin regions. Interestingly, these interactions appear to be independent of Treg cell development. The looping of DNA segments depicts the chromatin architecture that supports Foxp3 enhancers' function in regulating transcription.

### Example 3: Deficiencies of Enhancers CNS0/CNS3 and CNS0/CNS2 Cause Fatal Autoimmunity

To reveal the biological significance of the coordination of Foxp3 enhancers, CNS0/CNS3 and CNS0/CNS2 double-deficient (ΔCNS0,3 and ΔCNS0,2, respectively) mice were generated, which expressed the same GFP-Foxp3 fusion protein as WT Foxp3GFP. In addition, single-deficient mice were produced for comparison. Heterozygous female ΔCNS0,2 and ΔCNS0,3 mice were indistinguishable from WT mice, and hemizygous male ΔCNS0,2 and ΔCNS0,3 mice were born at Mendelian ratios equal to those of WT littermates. ΔCNS0,3 mice had a lifespan as short as Foxp3^{null} mice, indicating abolished Treg cell-mediated immune tolerance. ΔCNS0,2 mice died at age 2-4 months, whereas no fatality was observed in ΔCNS0 and ΔCNS2 mice up to 7 months, consistent with previous reports (Feng et al. (2014) Cell 158:749-763; Li et al. (2014) Cell 158:734-748). The longer lifespan of ΔCNS0,2 mice than that of Foxp3^{null} mice implies that ΔCNS0,2 leads to residual Treg cell function. Overall, the synthetic lethal autoimmunity of ΔCNS0,2 and ΔCNS0,3 indicates that these enhancers have significant overlapping or compensatory roles such that their single deficiencies are largely tolerated.

Consistent with survival, ΔCNS0,3 mice exhibited an aggressive autoimmune syndrome comparable to that of Foxp3^{null} mice at an early age (5 weeks old). In contrast, relatively milder inflammation was observed in 3- to 4-month-old ΔCNS0,2 mice. Despite different onsets, both ΔCNS0,2 and ΔCNS0,3 mice developed severe lymphoproliferative disease. Conventional T (Tcon) cells were markedly activated in these mice characterized by mixed T helper 1 (Th1; IFNγ⁺), Th2 (IL-13⁺), and Th17 (IL-17⁺) responses. To test whether Treg cells in ΔCNS0,2 mice suppress any specific autoimmunity, single-cell RNA sequencing (scRNA-seq) of the lymphocytes isolated from the spleen and lung of WT and ΔCNS0,2 mice was performed. This analysis revealed widespread immune activation as observed in Treg cell-deficient mice (Sakaguchi et al. (2020) Annu. Rev. Immunol. 38:541-566). Therefore, ΔCNS0,2 appears to nonselectively impair Treg cell function.

Systemic, fatal autoimmune inflammation in ΔCNS0,2 and ΔCNS0,3 mice demonstrates the significance of the coordination of Foxp3 enhancers, revealing that almost the entire Treg cell suppressive capacity relies on synergistic Foxp3 enhancers.

### Example 4: An Additive Effect of CNS0 and CNS3 on Thymic Treg Cell Development and Treg Cell TCR Diversity

The CRISPR screening result presented herein indicates that CNS0 and CNS3 coordinate to promote Foxp3 induction. To determine its role in Treg cell development in vivo, Treg cells in 3- to 4-week-old male mice were examined. ΔCNS0 or ΔCNS3 resulted in a two- to three-fold reduction of thymic Treg cell frequencies compared with WT mice. In contrast, ΔCNS0,3 decreased thymic Treg cells by approximately 10-fold, indicating a substantial cooperation between CNS0 and CNS3.

Reduced Treg cell induction in ΔCNS0 or ΔCNS3 mice was followed by elevated proliferation of Treg cells in the periphery through homeostatic expansion (Dikiy et al. (2021) Immunity 54:931-946.e11; Feng et al. (2015) Nature 528:132-136). In addition, inflammation also drives Treg cell proliferation (Kim et al. (2007) Nat. Immunol. 8:191-197). However, in the presence of both homeostatic and inflammatory drivers, ΔCNS0,3 Treg cells in the periphery remained significantly less than those in WT, ΔCNS0, or ΔCNS3 mice. If ΔCNS0,3 predominantly affected Foxp3 induction, Treg cell expansion was likely constrained by available antigens, which is comparable to the limited Treg cells derived from monoclonal transgenic TCRs against self-antigens (Buenrostro et al. (2013) Nat. Methods 10:1213-1218). Therefore, Treg cell clones for many self-antigens might be absent in ΔCNS0,3 mice.

To assess the cell-intrinsic effects of ΔCNS0,3, a competitive setting was generated with mixed bone marrow chimeric mice in which congenically marked WT Treg cells suppressed aberrant immune activation. ΔCNS0 or ΔCNS3 reduced thymic Treg cell frequencies by three- to four-fold. Remarkably, ΔCNS0,3 caused a more than 50-fold reduction of thymic Treg cells, indicating a strong synergistic effect of ΔCNS0 and ΔCNS3. ΔCNS0,3 Treg cells expressed higher Ki67, and their abundance in the periphery mildly increased relative to that in the thymus, indicating that ΔCNS0,3 Treg cells were more self-reactive as also observed in ΔCNS0 and ΔCNS3 Treg cells (Dikiy et al. (2021) Immunity 54:931-946.e11; Feng et al. (2015) Nature 528:132-136). Thus, Foxp3 induction in precursor cells bearing lower-affinity TCRs for self-antigens might be preferentially affected by ΔCNS0,3. Notably, ΔCNS0,3 reduced Foxp3 expression levels per cell by ˜50%, indicating that CNS0 and CNS3 play redundant roles in maintaining high levels of Foxp3 expression. ΔCNS0,3 also decreased CD25 expression, likely due to attenuated Foxp3-mediated positive regulation (Gavin et al. (2007) Nature 445:771-775) and/or activation-dependent downregulation of CD25 (van der Veeken et al. (2016) Cell 166:977-990).

Thymic Treg cell development appears to undergo two steps: TCR agonists triggering CD25 expression followed by IL-2 signaling-dependent Foxp3 induction, (Lio & Hsieh (2008) Immunity 28:100-111). CD4⁺CD8⁻CD25⁺Foxp3⁻ thymocytes were significantly accumulated in ΔCNS0,3 mice, indicating that ΔCNS0,3 blocks IL-2-induced precursor-to-Treg cell transition. This defect may convert many Treg precursor cells to autoreactive T effector cells. However, CD4 T effector cells in WT GFP, ΔCNS0, ΔCNS3, and ΔCNS0,3 compartments were equally suppressed by WT Treg cells. Therefore, the lethal autoimmunity in ΔCNS0,3 mice was primarily caused by the dysregulation of Foxp3 expression.

To understand to what extent the coordination between CNS0 and CNS3 determines the diversity of Treg cell TCR repertoire, the TCRα and TCRβ chains of Treg cells isolated from the lymphoid organs of age-matched male WT, ΔCNS0, ΔCNS3, and ΔCNS0,3 mice were profiled. A 0.7- to two-fold reduction of the diversity intensities (inverse Simpson indexes) of ΔCNS0 and ΔCNS3 Treg cells was observed. Similarly, ΔCNS0 and ΔCNS3 reduced the total unique TCRs of Treg cells in each animal. Markedly, ΔCNS0,3 decreased the diversity intensities and total unique TCRs of Treg cells by more than 40-fold. Given the varying immunological consequences of ΔCNS0, ΔCNS3, and ΔCNS0,3, this TCR sequencing result defines the dynamic range of the Treg cell TCR diversity related to nearly the entire spectrum of Treg cell suppressive capacity determined by CNS0 and CNS3, individually and collectively.

### Example 5: Additive Effects of CNS0 and CNS3 on Treg Cell Induction In Vitro and Treg Cell Lineage Stability

The effects of ΔCNS0, ΔCNS3, and ΔCNS0,3 on iTreg cell induction was subsequently determined. To avoid the influence of autoimmune inflammation, CD4 Tn cells were isolated from mixed bone marrow chimeric mice described above. A mild additive effect of ΔCNS0 and ΔCNS3 on iTreg cell induction was observed (FIG. 3), likely because of prolonged exposure to Treg cell-inducing cues (Materials and methods). Unlike nTreg cells, Foxp3 expression levels in iTreg cells were not influenced by the additive effect of ΔCNS0 and ΔCNS3.

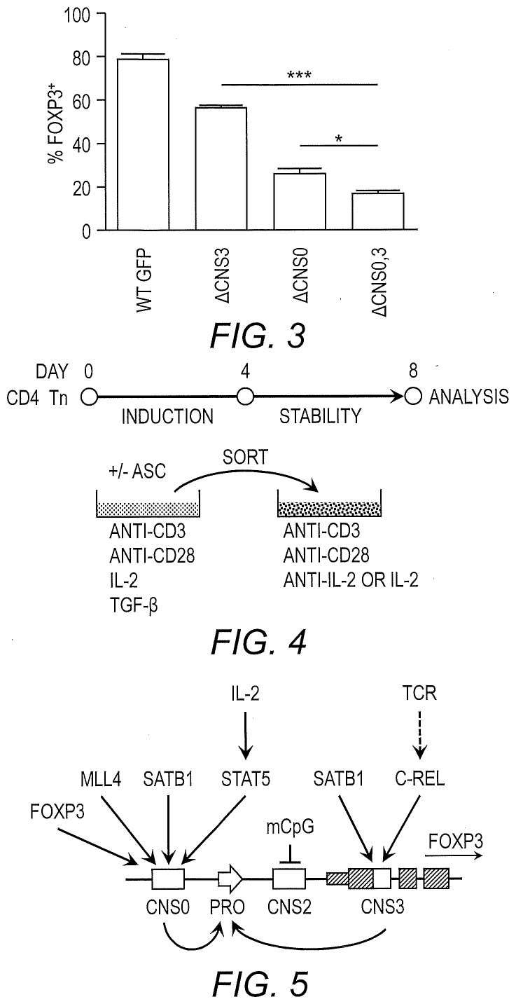

To test whether ΔCNS0,3 could also destabilize Foxp3 expression, the stability of Foxp3 expression was assessed in control and ASC-treated iTreg cells, which stabilize Foxp3 expression through Tet-dependent DNA demethylation (Yue et al. (2016) J. Exp. Med. 213:377-397). CD4 Tn cells from mixed bone marrow chimeras were used to induce iTreg cells with or without supplemented ASC in culture media. Subsequently, iTreg cells were sorted and cultured in fresh media without TGF-β for 4 days before analysis (FIG. 4). Without ASC treatment, high levels of IL-2 maintained Foxp3 expression in 40%-50% WT and ΔCNS3 but not in ΔCNS0 or ΔCNS0,3 iTreg cells, consistent with the role of CNS0 in maintaining Foxp3 expression before DNA demethylation (Dikiy et al. (2021) Immunity 54:931-946.e11). When IL-2 was blocked by neutralization antibodies, Foxp3 expression was silenced in more than 80% of all iTreg cells. Upon ASC treatment, iTreg cells of all the genotypes stabilized Foxp3 expression, despite minor differences, indicating that ΔCNS0,3 did not affect Tet-dependent stabilization of Foxp3 expression.

Next, the stability of Foxp3 expression in nTreg cells isolated from aged-matched male mice was assessed by cotransferring them with CD45.1⁺ WT CD4 Toon and Treg cells into Rag1^{-/-} mice. The cells were recovered to analyze Foxp3 expression 3-4 weeks later. In this adverse environment, 5%-20% of WT, ΔCNS0, and ΔCNS3 nTreg cells and 40%-60% of ΔCNS0,3 nTreg cells lost Foxp3 expression, indicating that ΔCNS0,3 nTreg cells had a defect in maintaining Foxp3 expression. Because nTreg cells in ΔCNS0,3 mice had undergone extensive expansion and had been exposed to high levels of proinflammatory cytokines, both destabilizing Foxp3 expression (Feng et al. (2014) Cell 158:749-763; Komatsu et al. (2014) Nat. Med. 20:62-68; Zhou et al. (2009) Nat. Immunol. 10:1000-1007), the effect of ΔCNS0,3 was likely overestimated in this assay.

To assess the effect of ΔCNS0,3 on nTreg cell function on a per-cell basis, Treg cell in vitro suppression assays were performed with nTreg cells sorted from aged-matched male WT GFP and ΔCNS0,3 mice. In the presence of invariant TCR agonists (i.e., anti-CD3 antibodies), ΔCNS0,3 nTreg cells exhibited significantly stronger suppression of CD4 Toon cell proliferation than WT nTreg cells did. To test if this result was caused by the preexposure of nTreg cells to inflammatory conditions in ΔCNS0,3 mice, similar assays were performed with nTreg cells sorted from WT GFP and ΔCNS0,3 mixed bone marrow chimeric mice. ΔCNS0,3 nTreg cells displayed a milder, but significantly higher suppressive activity. This result was consistent with elevated activation of ΔCNS0,3 nTreg cells in mixed bone marrow chimeras. These data indicate that ΔCNS0,3 does not dramatically impair Treg cell function per cell, despite reduced Foxp3 expression level and stability.

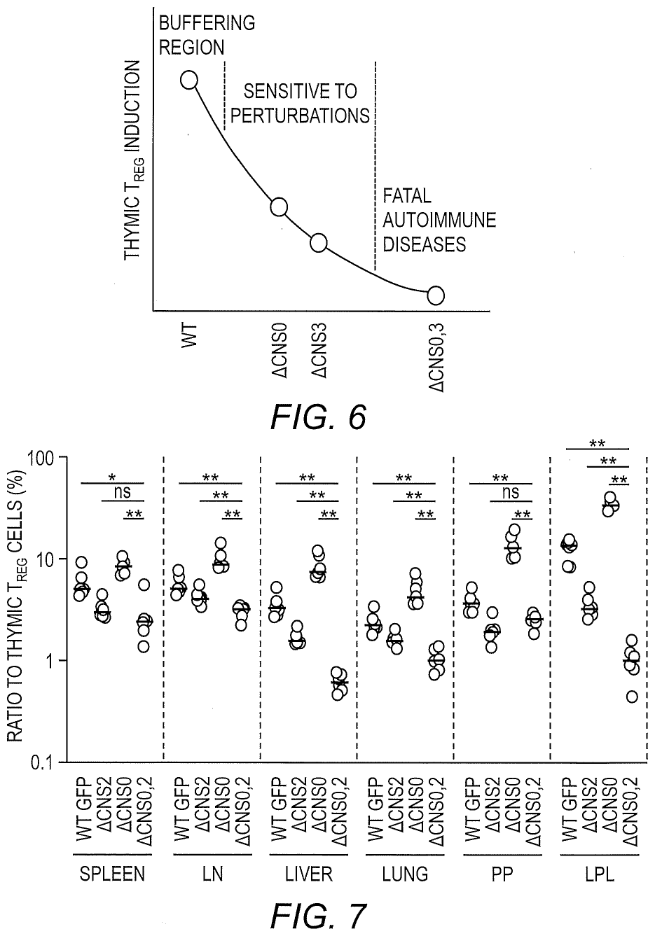

CNS0 and CNS3 coordinate to promote Foxp3 induction efficiency, expression level, and maintenance, among which Foxp3 induction is affected most by ΔCNS0,3 in terms of the fold changes. Analyses indicated that reduced Treg cell development decreases the representation of TCR clones for certain epitopes in ΔCNS0 or ΔCNS3 mice. Although this defect is insufficient to cause immune dysregulation, it sensitizes ΔCNS0 and ΔCNS3 mice to other impaired tolerance mechanisms, e.g., Aire deficiency (Dikiy et al. (2021) Immunity 54:931-946.e11; Feng et al. (2015) Nature 528:132-136). Further reduction of Treg cell development by ΔCNS0,3 may deplete Treg cell clones for all the epitopes of many self-antigens, resulting in severe autoimmune inflammation. Mechanistically, CNS3 and CNS0 integrate TCR (Feng et al. (2015) Nature 528:132-136; Zheng et al. (2010) Nature 463:808-812) and IL-2/Stat5 signaling (Dikiy et al. (2021) Immunity 54:931-946.e11), respectively, to enhance Foxp3 expression (FIG. 5). Involvement of additional factors (Feng et al. (2015) Nature 528:132-136; Kitagawa et al. (2017) Nat. Immunol. 18:173-183; Placek et al. (2017) Nat. Immunol. 18:1035-1045) indicates that the coordination of CNS0 and CNS3 relies on multiple molecular mechanisms. Overall, these two distinct regulatory modules cooperate to maximize Treg cell suppressive capacity (FIG. 6).

### Example 6: CNS0 and CNS2 Coordinate to Confer Stable Treg Cell Lineage Identity

CNS2 maintains heritable Foxp3 expression, and CNS0 facilitates Foxp3 induction, raising a possibility of their combined defects in causing fatal autoimmunity in ΔCNS0,2 mice. However, CRISPR screening results indicate that CNS0 and CNS2 may coordinate to maintain Foxp3 expression (FIG. 2). To distinguish between these two models, Treg cells in 3- to 4-week-old mice were examined. Comparable reduction of thymic Treg cells in ΔCNS0 and ΔCNS0,2 mice was observed. In the periphery, homeostatic expansion largely compensated for the Treg cell developmental defect in ΔCNS0 mice but not in ΔCNS0,2 mice. This result, together with decreased Foxp3 protein per cell and constrained activation (shown by CD44, CTLA-4, and Ki67 expression) of ΔCNS0,2 Treg cells in the presence of inflammation, indicates that CNS0 and CNS2 coordinate to maintain Foxp3 expression during activation-dependent proliferation.

To reveal the cell-intrinsic effects of ΔCNS0,2, chimeric mice were generated with CD45.2 WT GFP, ΔCNS0, ΔCNS2, or ΔCNS0,2, and CD45.1 WT bone marrow. ΔCNS0 and ΔCNS0,2 led to a similar reduction in thymic Treg cells. Unlike ΔCNS0 Treg cells, ΔCNS0,2 Treg cells remained at lower frequencies in the periphery. When the extent of expansion was estimated by the ratios of peripheral-to-thymic Treg cell numbers in individual mice, ΔCNS0 Treg cells expanded significantly more than WT Treg cells did (FIG. 7). In contrast, ΔCNS2 Treg cells expanded considerably less, which was further reduced in ΔCNS0,2 mice. As ΔCNS0,2 Treg cells expressed lower levels of Foxp3, CTLA-4, CD44, and Ki67, their reduced peripheral expansion indicates a crucial role of CNS0,2's coordination in maintaining Treg cell identity.

Treg cell lineage stability was subsequently assessed. To alleviate the potential influence of different activation statuses, equal numbers of activated) (CD44^{hi}CD62L^{lo}) and resting (CD44^{lo}CD62L^{hi}) Treg cells for each genotype were transferred into Rag1^{-/-} mice. Four weeks later, 10%-30% of WT and ΔCNS0 nTreg cells and 40%-60% of ΔCNS2 Treg cells lost Foxp3 expression. In contrast, more than 90% ΔCNS0,2 Treg cells silenced Foxp3 expression, demonstrating an essential role of CNS0,2's coordination in maintaining Treg cell identity. Impaired Treg cell function upon Foxp3 loss, together with the effector-like exTreg cells (or “former” Treg cells; Feng et al. (2014) Cell 158:749-763; Komatsu et al. (2014) Nat. Med. 20:62-68; Zhou et al. (2009) Nat. Immunol. 10:1000-1007), may contribute to the progressive, lethal autoimmune lesions in ΔCNS0,2 mice.

### Example 7: scRNA-Seg Reveals the Functional States of ΔCNS0,2 Treg Cells

To assess Treg cell lineage stability and functional states in a competitive, lymphocompetent environment, scRNA-seq was performed with Treg cells sorted from mixed bone marrow chimeric mice, which revealed 17 Seurat clusters associated with differentially expressed genes (Table 1).

TABLE 1

| I (C0) | | II (C1) | |
|---|---|---|---|
| Ly6c1 | Satb1\hat{ } | Cst7 | Rilpl2 |
| Igfbp4 | Pdlim1 | Cd83 | Tcf7* |
| Klf2\hat{ } | Txnip\hat{ } | Nrn1 | Zfp3611 |
| Sell\hat{ } | Rps7 | 2310001H17Rik | Hif1a* |
| Ms4a4b\hat{ } | Rps20 | Bhlhe40* | Tnfsf8 |
| Rps19 | Gm2682 | Tiam1 | Id3 |
| Bcl2 | Rexo2 | Tbc1d4 | Ephx1 |
| Rps11 | Rps18 | Sh2d1a | Cd82 |
| Gpb2 | S1pr1 | Izumo1r | Nfatc1 |
| Rpl12 | Rpl8 | Tnfsf11 | Lax1 |
| III (C2) | | IV (C3) | |
| Tigit* | Tnfrsf1b | Trbv12-1 | Rpl10a |
| Icos* | Matk | Gsta4 | Rpl36a |
| Ccl5* | Serpina3g | Ccr7\hat{ } | Rpl32 |
| AW112010 | Lag3* | Gpr83 | Igf2r |
| Ly6a | Ctla4* | H2afz | Ift80 |
| Il10* | S100a6 | I12ra\hat{ } | Rpl35 |
| Glrx | Tnfrsf4 | Nsg2 | Chchd10 |
| Tnfrsf9 | Neb | Rps26 | Itgb1 |
| Maf | Cxcr3* | Ecm1 | Nrp1 |
| S100a11 | Srgn | Mbnl3 | Gm14085 |
| V (C4) | | VI (C5) | |
| I17r | Ifngr1 | Mif | Set |
| Zfp36l2 | Rflnb | Ncl | Nop58 |
| Klf3 | Ms4a6b | Srm | Eif4a1 |
| Evl | Klhl6 | Pa2g4 | Apex1 |
| 2810474O19Rik | Ssbp2 | Eif5a | Hsp90ab1 |
| Samhd1 | Socs3 | C1qbp | Mybbp1a |
| Smc4 | Dgka | Nhp2 | Atp5g1 |
| | | Hspd1 | Npm1 |
| | | Fbl | Ccr2 |
| | | Ran | |
| VII (C6) | | VIII (C7) | |
| Gbp2b | Ccr4* | Lgals1 | Tagln2 |
| Ccr9 | Tmem176b | Crip1 | Ifi2712a |
| Itm2b | Socs2* | S100a10* | Ahnak |
| S100a4 | Jun* | Vim* | Gmfg |
| Prg4 | Sdc4 | Anxa2 | Cdc25b |
| Emb | Rorc* | Ass1 | Flna |
| IX (C8) | | X (C9) | |
| Actg1 | Dhcr24 | Srebf2 | Rnf213 |
| Hmgcs1 | Insig1 | Isg15 | Slfn5 |
| Ldlr | Idi1 | Ifit1 | Ifi208 |
| Fdft1 | Hmgcr | Ifit3 | Slfn1 |
| Cyp51 | Rasgrp2 | Irf7 | Trim30a |
| Sqle | Emp3 | Bst2 | Phf11b |
| Msmo1 | Mvd | Usp18 | Ifi209 |
| Fdps | | Rtp4 | Xaf1 |
| | | Zbp1 | Ili213 |
| | | Isg20 | Daxx |
| XI (10) | | XII (C11) | |
| Malat1 | Phf2011 | Pcif1 | Ea1 |
| mt-Cytb | Vdac2 | Smim14 | Cyfip1 |
| mt-Nd4l | Papola | Il1r2 | Tpi1 |
| mt-Co2 | Bcl10 | Pdcd1 | Aldoa |
| mt-Co1 | Top1 | Igkc | Stx11 |
| Dctn3 | Pak2 | Smco4 | Gna13 |
| Fermt3 | Gm2a | Gm30211 | Cxcr5 |
| Ubac2 | Plekhj1 | Gm42031 | Rgs16 |
| Ripor2 | Uqcrfs1 | Sccpdh | |
| XIII (C12) | | XIV (C13, C14) | |
| Sostdc1 | Cd40lg | Stmn1 | Lmnb1 |
| Dapl1 | Gm45552 | Pclaf | Hmgn2 |
| Gm20400 | Itm2a | Ube2c | Tubb4b |
| Lef1 | Gtf2i | Hmgb2 | Smc2 |
| Actn1 | | Top2a | Cdca8 |
| | | Birc5 | Asf1b |
| | | H2afx | Cks2 |
| | | Tuba1b | Cenpa |
| | | Tubb5 | Ccnb2 |
| | | Mki67 | Selenoh |
| | | Rrm2 | Cks1b |
| | | Ccna2 | H2afv |

\hat{ }Gene in resting state marker
*Gene in activation state marker

Among them, substantially fewer cells were identified in activation-related clusters in ΔCNS2 and ΔCNS0,2 Treg cells than in WT and ΔCNS0 counterparts. Pseudotime is widely used to reconstruct the dynamic processes experienced by cells in a heterogeneous population (Trapnell et al. (2014) Nat. Biotechnol. 32:381-386). Applying this algorithm to Treg cells revealed that ΔCNS0,2 mainly impaired Treg cells at the terminally activated state (state S4), whereas Treg cells at the resting (S1 and S7) and intermediate (S2, S3, S5, and S6) states were less affected, if any. Together with impaired Treg cell stability observed in the in vivo assay, these results indicate that ΔCNS0,2 mainly disrupted Treg cell lineage stability during activation-dependent clonal expansion.

To demonstrate this, TCR clonotypes were profiled using scRNA-seq. Although only 8,000-10,000 Treg cells were sampled, this experiment uncovered profound effects of Foxp3 enhancer deficiencies on the clonotypes of Treg cell TCRs without influencing their usages of V, D, and J gene segments; while ΔCNS0 Treg cells exhibited elevated clonal expansion (˜5% clones with >1 cell), ΔCNS2 and ΔCNS0,2 Treg cells had markedly fewer duplicate TCRs (˜1% clones with >1 cell, compared to ˜3% clones with >cell in the WT GFP control). This result was strikingly consistent with the expansion potential of Treg cells estimated in mixed bone marrow chimeras. Treg cells with multiple copies were located in the clusters expressing higher levels of activation markers, e.g., Tigit, Ctla-4, IL-10, and Cxcr3. These clusters were depleted in ΔCNS2 and ΔCNS0,2 Treg cells. The scRNA-seq results support the crucial role of CNS0,2's coordination in maintaining Treg cell identity during activation-dependent expansion.

### Example 8: Epistasis Between CNS0/CNS2 and the Epigenetic Programs Controlling Foxp3 Expression

Whereas CNS2's function is activated by Tet-mediated DNA demethylation (Yue et al. (2016) J. Exp. Med. 213:377-397), CNS0 does not show differential DNA methylation during Treg cell development. Stat5 binds to CNS0 upon IL-2 stimulation in both differentiating and mature Treg cells (Dikiy et al. (2021) Immunity 54:931-946.e11). The dramatic effects of ΔCNS0,3 and ΔCNS0,2 indicate that CNS0 contributes to both Treg cell development and Treg cell lineage stability, with the latter being shielded by CNS2's function in WT Treg cells.

Tet-dependent DNA demethylation is a key mechanism securing Foxp3 expression (Sakaguchi et al. (2020) Annu. Rev. Immunol. 38:541-566). As IL-2/Stat5 signaling was proposed to recruit Tet enzymes (Yang et al. (2015) Immunity 43:251-263), CNS0 and CNS2 may act via a partially redundant mechanism to promote this process. To demonstrate this, whole-genome bisulfite sequencing (WGBS) was performed with Treg cells isolated from mixed bone marrow chimeric mice that were derived from male donors because the Foxp3 gene is located on the X chromosome. Treg cell-specific DNA demethylation around the Foxp3 locus was confirmed. Surprisingly, except for some upstream, less conserved regions, ΔCNS0,2 did not cause significant DNA hypermethylation in the remaining Treg cell-specific demethylated regions downstream of Foxp3 promoter, indicating that Tet recruitment or enzymatic activity at these regions is independent of the two major IL-2/Stat5 response elements.

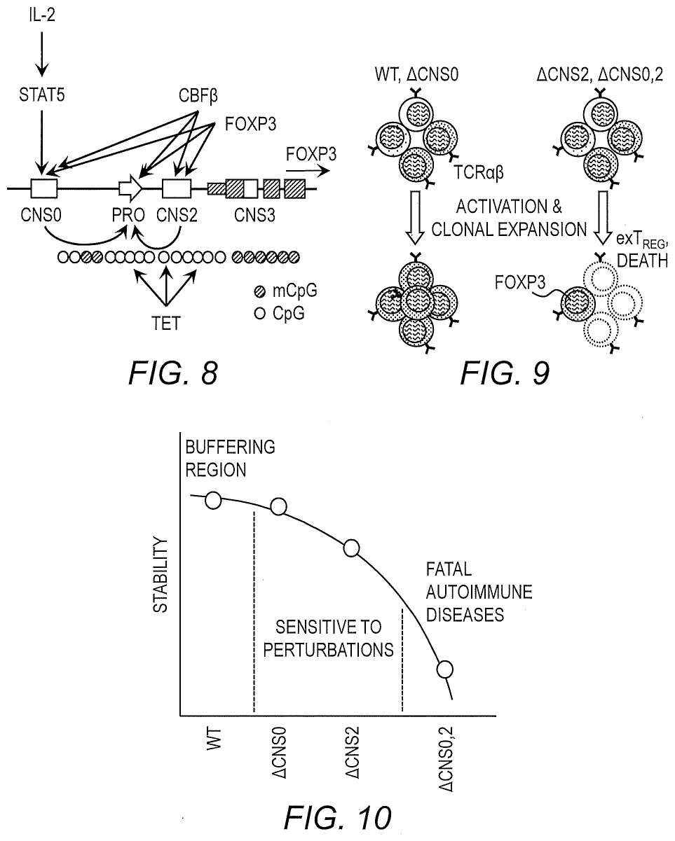

To understand how CNS0 and CNS2 coordinate to stabilize Foxp3 transcription, active promoter and enhancer markers were examined with CUT&RUN sequencing (Skene & Henikoff (2017) eLife 6:e21856). Differences of H3K4me3 and H3K27ac at the Foxp3 locus were not observed in WT, ΔCNS0, ΔCNS2, and ΔCNS0,2 nTreg cells, although RNA polymerase II (Pol II) association was drastically reduced in ΔCNS0,2 nTreg cells. As ΔCNS0, ΔCNS2, and ΔCNS0,2 did not change the local chromatin accessibility as assessed by assay for transposase-accessible chromatin using sequencing (ATAC-seq), CNS0 and CNS2 likely stabilize Foxp3 transcription through their associated factors (FIG. 8).

Despite nonoverlapping roles, distinct epigenetic regulation, and temporal activities, CNS0 and CNS2 coordinate to maintain Treg cell lineage identity, especially during activation and expansion, conferring persistent immune tolerance (FIG. 9-10). This process appears to be independent or downstream of the mechanisms governing the deposition of permissive histone markers and DNA demethylation.

What is claimed is:

1. A method for modifying regulatory T cell lineage stability comprising simultaneously modulating the activity of conserved noncoding sequence (CNS) 0 and CNS2 of the forkhead box P3 (Foxp3) locus in a regulatory T cell.

2. The method of claim 1, wherein regulatory T cell lineage is destabilized.

3. The method of claim 2, wherein the activity of CNS0 and CNS2 is reduced.

4. The method of claim 3, wherein a reduction of CNS0 and CNS2 activity is via one or more protein factors that bind CNS0 and CNS2, CNS0 and CNS2 silencing, or inhibiting chromatin looping of CNS0 and CNS2 with the Foxp3 promoter.

5. The method of claim 4, wherein CNS0 and CNS2 are silenced by recruiting transcriptional or epigenetic inhibitory protein factors to CNS0 and CNS2 via CRISPR-based or TALEN-based approaches.

6. The method of claim 1, wherein regulatory T cell lineage stability is enhanced.

7. The method of claim 6, wherein the activity of CNS0 and CNS2 is increased.

8. The method of claim 7, wherein an increase in CNS0 and CNS2 activity is via one or more protein factors that bind CNS0 and CNS2, recruiting transcriptional or epigenetic activating protein factors to CNS0 and CNS2 via CRISPR-based or TALEN-based approaches, insertion of at least one exogenous CNS0 and CNS2 nucleic acid molecule into the Foxp3 locus, or by increasing chromatin looping of CNS0 and CNS2 with the Foxp3 promoter.

9. A method for treating cancer comprising
(a) reducing the activity of conserved noncoding sequence (CNS) 0 and CNS2 in a regulatory T cell; and
(b) administering the regulatory T cell of (a) to a subject with cancer, thereby treating the subject's cancer.

10. The method of claim 9, wherein a reduction of CNS0 and CNS2 activity is via one or more protein factors that bind CNS0 and CNS2, CNS0 and CNS2 silencing, or inhibiting chromatin looping of CNS0 and CNS2 with the Foxp3 promoter.

11. The method of claim 10, wherein CNS0 and CNS2 are silenced by recruiting transcriptional or epigenetic inhibitory protein factors to CNS0 and CNS2 via CRISPR-based or TALEN-based approaches.

12. A method for treating an autoimmune disease or condition comprising
(a) increasing the activity of conserved noncoding sequence (CNS) 0 and CNS2 in a regulatory T cell; and
(b) administering the regulatory T cell of (a) to a subject with an autoimmune disease or condition, thereby treating the subject's autoimmune disease or condition.

13. The method of claim 12, wherein an increase in CNS0 and CNS2 activity is via one or more protein factors that bind CNS0 and CNS2, recruiting transcriptional or epigenetic activating protein factors to CNS0 and CNS2 via CRISPR-based or TALEN-based approaches, insertion of at least one exogenous CNS0 and CNS2 nucleic acid molecule into the Foxp3 locus, or by increasing chromatin looping of CNS0 and CNS2 with the Foxp3 promoter.

14. A method for modifying regulatory T cell induction comprising simultaneously modulating the activity of conserved noncoding sequence (CNS) 0 and CNS3 in a regulatory T cell precursor.

15. The method of claim 14, wherein regulatory T cell precursor comprises a naïve CD4 T cell, hematopoietic stem cell, or lymphoid progenitor cell.

16. The method of claim 14, wherein the activity of CNS0 and CNS3 is increased.

17. The method of claim 16, wherein an increase in CNS0 and CNS3 activity is via one or more protein factors that bind CNS0 and CNS3, recruiting transcriptional or epigenetic activating protein factors to CNS0 and CNS3 via CRISPR-based or TALEN-based approaches, insertion of at least one exogenous CNS0 and CNS3 nucleic acid molecule into the Foxp3 locus, or by increasing chromatin looping of CNS0 and CNS3 with the Foxp3 promoter.

18. A method for treating a regulatory T cell-related disease or condition caused by impaired regulatory T cell diversity or coverage comprising
(a) increasing the activity of conserved noncoding sequence (CNS) 0 and CNS3 in a regulatory T cell precursor; and
(b) administering the regulatory T cell precursor of (a) to a subject in need of treatment thereby treating the subject's regulatory T cell-related disease or condition caused by impaired regulatory T cell diversity or coverage.

19. The method of claim 18, wherein regulatory T cell precursor comprises a naïve CD4 T cell, hematopoietic stem cell, or lymphoid progenitor cell.

20. The method of claim 18, wherein an increase in CNS0 and CNS3 activity is via one or more protein factors that bind CNS0 and CNS3, recruiting transcriptional or epigenetic activating protein factors to CNS0 and CNS3 via CRISPR-based or TALEN-based approaches, insertion of at least one exogenous CNS0 and CNS3 nucleic acid molecule into the Foxp3 locus, or by increasing chromatin looping of CNS0 and CNS3 with the Foxp3 promoter.


SEQUENCE LISTING (13 sequences)

SEQ ID NO: 1 - DNA, 500 bp, Homo sapiens
gaaaagggatcccctgaggtccaccaccatttccccagagggctggatcacggggggtagctattcttcaacagcacttcaaatcagcagcagcacacaggccttaaaacaataataagttgaaatgtatttgctaggaaagtcaccgacctacaaagaaaaccttatcgctgatctagcagcgcacaccagcctcccctttgcaagagctgagatcaaaagataaagaagctatcaaaaagccatctgcccacttaaaataacatctcaagtcacgttgggaaccacaaacatggggccagctaccaaaacaattgtctaaatgaactacttcaatttctccttaaaaccacccatgtattttaaaagaaaaacaccctctccacccaccttggcacggcaaggttttgatttgtctgttcccttcctttcacattcttgaaaatgaccaaacttcagtactcaactgtcttatcttccagaaagggctcccacaactg

SEQ ID NO: 2 - DNA, 519 bp, Homo sapiens
gggttttgcatggtagccagatggacgtcacctaccacatccgctagcacccacatcaccctacctgggcctatccggctacaggatagactagccacttctcggaacgaaacctgtggggtagattatctgcccccttctcttcctccttgttgccgatgaagcccaatgcatccggccgccatgacgtcaatggcagaaaaatctggccaagttcaggttgtgacaacagggcccagatgtagaccccgataggaaaacatattctatgtcccagaaacaacctccatacagcttctaagaaacagtcaaacaggaacgccccaacagacagtgcaggaagctggctggccagcccagccctccaggtccctagtaccactagacagaccatatccaattcaggtcctctttctgagaatgtactgatgcatcacacagtcacaccagttccacaagtatttaaggaggagatttcttataagttctgaccaaacataaagagcacttcaaaagtga

SEQ ID NO: 3 - DNA, 224 bp, Homo sapiens
gtgaggcccggggcccagaatggggtaagcagggtggggtacttgggcctataggtgtcgacctttactgtggcatgtggcgggggggggggggggggctggggcacaggaagtggtttatgggtcccaggcaagtctgacttatgcagatattgcagggccaagaaaatccccactctccaggcttcagagattcaaggctttccccacccctcccaatcctc

SEQ ID NO: 4 - DNA, 17 bp, Artificial Sequence
  Synthetic oligonucleotide
agtcaaagtcggtgaac

SEQ ID NO: 5 - DNA, 16 bp, Artificial Sequence
  Synthetic oligonucleotide
atctctgcttttgatg

SEQ ID NO: 6 - DNA, 42 bp, Artificial Sequence
  Synthetic oligonucleotide
  misc_feature (21)..(24): n is a, c, g, t or u
  misc_feature (26)..(29): n is a, c, g, t or u
  misc_feature (31)..(34): n is a, c, g, t or u
caguggtaucaacgcagagunnnnunnnnunnnnucttgggg

SEQ ID NO: 7 - DNA, 33 bp, Artificial Sequence
  Synthetic oligonucleotide
cactctatccgacaagcagtggtatcaacgcag

SEQ ID NO: 8 - DNA, 20 bp, Artificial Sequence
  Synthetic oligonucleotide
cactctatccgacaagcagt

SEQ ID NO: 9 - DNA, 20 bp, Artificial Sequence
  Synthetic oligonucleotide
gctgtcctgagaccgaggat

SEQ ID NO: 10 - DNA, 20 bp, Artificial Sequence
  Synthetic oligonucleotide
atggctcaaacaaggagacc

SEQ ID NO: 11 - DNA, 28 bp, Artificial Sequence
  Synthetic oligonucleotide
  misc_feature (1)..(2): Nucleotide may be present or absent
  misc_feature (3)..(9): n is a, c, g, or t
nnnnnnnnncagtggtatcaacgcagag

SEQ ID NO: 12 - DNA, 30 bp, Artificial Sequence
  Synthetic oligonucleotide
  misc_feature (1)..(2): Nucleotide may be present or absent
  misc_feature (3)..(9): n is a, c, g, or t
nnnnnnnnncaggttctgggttctggatgt

SEQ ID NO: 13 - DNA, 29 bp, Artificial Sequence
  Synthetic oligonucleotide
  misc_feature (1)..(2): Nucleotide may be present or absent
  misc_feature (3)..(9): n is a, c, g, or t
nnnnnnnnnagtcacatttctcagatcct